(19) United States
(12) Patent Application Publication
(10) Pub. No.: US 2022/0328188 A1
(43) Pub. Date: Oct. 13, 2022

(54) ARTIFICIAL INTELLIGENCE METHODS FOR PREDICTING EMBRYO VIABILITY BASED ON MICROSCOPY METHODS

(71) Applicant: Optiva Fertility, Inc., Malden, MA (US)

(72) Inventor: Timothy H. SANCHEZ, Malden, MA (US)

(73) Assignee: Optiva Fertility, Inc., Malden, MA (US)

(21) Appl. No.: 17/712,593

(22) Filed: Apr. 4, 2022

Related U.S. Application Data
(60) Provisional application No. 63/170,180, filed on Apr. 2, 2021.

(51) Int. Cl. G16H 50/20; G16C 20/20; G16H 10/40; G06T 7/00; G01N 21/64

(52) U.S. Cl. CPC G16H 50/20; G16C 20/20; G16H 10/40; G06T 7/0012; G01N 21/6458; G06T 2207/30044; G06T 2207/10064; G06T 2207/10056; G06T 2207/20081; G06T 2207/20084

(57) ABSTRACT

Microscopy methods for determining embryo viability are described. A method can include accessing, at a compute device, fluorescence lifetime imaging microscopy (FLIM) data set associated with a biological material. The biological material can include either an embryo or a gamete. The method further includes extracting a fluorescence photon arrival time from a subset of data from the FLIM data set. The method further includes estimating a likelihood that the biological material will produce a successful pregnancy and/or a live birth based on the fluorescence photon arrival time histogram and an estimation model that has been trained using artificial intelligence and labeled clinical training data. The method includes generating an output signal representing the estimated likelihood that the biological material will produce a successful pregnancy and/or a live birth. In some embodiments, the method can include training the estimation model using a plurality of fluorescence photon arrival time histograms of the FLIM data set.

### CROSS-REFERENCE TO RELATED APPLICATIONS

This application claims priority to and the benefit of U.S. Provisional Patent Application No. 63/170,180, filed Apr. 2, 2021 and titled “Artificial Intelligence Methods for Predicting Embryo Viability Based on Microscopy Methods,” the disclosure of which is hereby incorporated by reference in its entirety.

### TECHNICAL FIELD

Embodiments described herein relate to microscopy methods for embryo viability prediction.

### BACKGROUND

Infertility affects an increasing number of couples. A significant portion of them turn to “In Vitro Fertilization” (IVF). IVF is a process that includes extracting eggs, retrieving a sperm sample, and then manually combining an egg and sperm in a laboratory dish to form one or more embryos. The embryos are then transferred to the uterus. While IVF is popular, its success rate remains relatively low. The inability to predict which embryos are most viable is a significant limitation of the IVF process, wherein viability is the chance of implantation and development to a live baby. Only about 10% of embryos obtained after IVF are able to implant after transfer in utero. Moreover, some patients have multiple embryos transferred in a single cycle to improve their odds of pregnancy. This practice can give rise to high rates of multiple gestations, with higher associated rates of mortality and suffering. Egg and embryo quality are important factors for determining the outcome of IVF procedures. For patients who use their own eggs, success rates decline steeply with maternal age due to degradation in egg quality. However, patients using donated eggs from young donors have the same chance of success as young patients. Better detection of viable embryos can increase the overall efficiency, safety, and effectiveness of IVF.

### SUMMARY

Embodiments described herein relate to use of microscopy in determining embryo viability. In some embodiments, a method can include accessing at a compute device, fluorescence lifetime imaging microscopy (FLIM) data set associated with a biological material. The biological material can include either an embryo or a gamete. The method further includes extracting a fluorescence photon arrival time from a subset of data from the FLIM data set. The method further includes estimating a likelihood that the biological material will produce a successful pregnancy and/or a live birth based on the fluorescence photon arrival time histogram and an estimation model that has been trained using artificial intelligence and labeled clinical training data. The method includes generating an output signal representing the estimated likelihood that the biological material will produce a successful pregnancy and/or a live birth. In some embodiments, the method can include training the estimation model using a plurality of fluorescence photon arrival time histograms of the FLIM data set. In some embodiments, the plurality of fluorescence photon arrival time histograms can include one of a raw intracellular FLIM histogram or a normalized intracellular FLIM histogram. In some embodiments, the method can further include combining multiple fluorescence photon arrival time histograms from the plurality of fluorescence photon arrival time histograms of the FLIM data set.

### BRIEF DESCRIPTION OF THE DRAWINGS

FIG. 1 is a block diagram of a method for predicting embryo viability, according to an embodiment.

FIG. 2 shows a demonstration of fluorescence in the context of microscopy.

FIG. 3 shows a pulsed illumination scheme, as implemented in the context of microscopy.

FIG. 4 is a photograph depicting a 1-photon fluorescence scheme and a 2-photon fluorescence scheme.

FIG. 5 shows diagrams illustrating time-domain FLIM as well as frequency-domain FLIM.

FIG. 6 a time series plot of fluorescence photon arrival times and a plot of photon counts versus fluorescence photon arrival times.

FIG. 7 shows a graphical illustration of time-gating FLIM.

FIG. 8 shows plots articulating differences between time-correlated single photon counting (TCSPC), time-gating, and pulse sampling.

FIG. 9 shows an example of an intracellular region and a series of plots showing a method for embryo viability prediction, in accordance with some embodiments.

FIG. 10 shows plots of fitted data for NADH and FAD, and an associated metabolic profile, in accordance with an embodiment.

FIG. 11 shows example synthetic fit data for a good embryo and a bad embryo.

FIG. 12 shows FLIM imagery for a single embryo, in accordance with an embodiment.

FIG. 13 shows FLIM imagery and associated fitted data for NADH, in accordance with an embodiment.

FIG. 14 includes metabolic parameter plots showing data separation between embryos resulting in pregnancy and embryos not resulting in pregnancy, in accordance with some embodiments.

FIG. 15A is a plot showing a logistic regression fit based on three metabolic parameters, for viability prediction, in accordance with an embodiment.

FIG. 15B is a receiver operating characteristic (ROC) curve showing true positive rate vs. false positive rate for the data of FIG. 15A.

FIG. 16 shows p-values for various multi-parameter models, for the data set of FIGS. 15A-15B.

FIG. 17 shows a comparison of ROC curves showing true positive rate vs. false positive rate for (1) viability predictions based on morphology, age, and pre-implantation genetic testing (PGT) status only, and (2) viability predictions based on metabolic parameters combined with the contextual parameters listed in (1), in accordance with some embodiments.

FIG. 18 includes metabolic parameter plots comparing discarded aneuploid vs. pilot embryos, in accordance with some embodiments.

FIG. 19A a plot showing a logistic regression fit based on three metabolic parameters, for differentiating between pregnant embryos and discarded aneuploid, in accordance with some embodiments.

FIG. 19B is a ROC curve showing true positive rate vs. false positive rate for the data of FIG. 19A.

FIG. 19C shows logistic regression results associated with FIG. 19A.

FIG. 20 shows image segmentation probability maps for intracellular pixels of a FLIM-imaged embryo, for various different probability thresholds, in accordance with an embodiment.

FIG. 21 includes plots comparing T-tests of pregnancy vs. non-pregnancy for each metabolic parameter, with variations in threshold, in accordance with an embodiment.

FIG. 22 includes plots of metabolic parameters vs. pregnancy outcomes, plots showing data separation among embryos resulting in pregnancy and embryos not resulting in pregnancy, using a 3-exponential function, in accordance with some embodiments.

FIG. 23 includes plots of human discarded oocyte (egg) metabolic parameters vs. patient age categories, in accordance with some embodiments.

FIG. 24A a plot showing a logistic regression fit for eggs from older vs. younger patients, in accordance with some embodiments.

FIG. 24B is a ROC curve showing true positive rate vs. false positive rate for the data of FIG. 24A.

FIG. 25 includes metabolic parameter plots showing data separation between discarded eggs having good vs. bad morphology, in accordance with some embodiments.

FIG. 26A a plot showing a logistic regression fit for discarded eggs having good vs. bad morphology, in accordance with some embodiments.

FIG. 26B is a ROC curve showing true positive rate vs. false positive rate for the data of FIG. 26A.

FIG. 27 shows example FLIM metabolic parameter images for each of FAD and NADH, in accordance with some embodiments.

FIG. 28 is a graphical depiction of channel stacking of the images of FIG. 27.

FIG. 29 shows various types of data augmentation that can be applied to original FLIM metabolic parameter images, in accordance with some embodiments.

FIG. 30A shows an example architecture of a neural network (NN) model, “EmbryoNet,” that represents fit parameters as channels in a third dimension, in accordance with an embodiment.

FIG. 30B shows an example architecture of a NN model, “EmbryoNet 3D,” that represents fit parameters as channels in a fourth dimension, in accordance with an embodiment.

FIG. 31A is an image of an example input to the EmbryoNet model, in accordance with an embodiment.

FIG. 31B is an image of an example input to the EmbryoNet 3D model, in accordance with an embodiment.

FIG. 32A shows an example architecture of a NN model with a fully connected (FC) head, “EmbryoNet-FC,” in accordance with an embodiment.

FIG. 32B is an image of an example input to the EmbryoNet-FC model, in accordance with an embodiment.

FIG. 33 shows training accuracy, validation accuracy, training loss, and validation loss plots for the EmbryoNet-FC model, in accordance with an embodiment.

FIG. 34 shows confusion matrices of results of the EmbryoNet-FC model on a pilot dataset, in accordance with an embodiment.

FIG. 35A shows an example architecture for a feature extraction (FE) model based on EmbryoNet (“EmbryoNet-FE”), in accordance with an embodiment.

FIG. 35B is an image of an example input to the FE model of FIG. 35A, in accordance with an embodiment.

FIG. 36 shows training accuracy, validation accuracy, training loss, and validation loss plots for the EmbryoNet-FE model, in accordance with an embodiment.

FIG. 37 shows confusion matrices of results of the EmbryoNet-FE model on a pilot dataset, in accordance with an embodiment.

FIG. 38 includes tabulated validation results on pilot data, comparing various models, showing fold accuracies for the highest-performing models.

### DETAILED DESCRIPTION

In vitro fertilization (IVF) includes selection of one or more viable embryos. Embryo quality and egg quality are important factors in determining the outcome of an IVF procedure. Hence, accurate methods of assessing egg and embryo quality and selecting the eggs and embryos with the highest chance of success has long been a central goal for assisted reproductive medicine. One method for non-invasively assessing embryo viability uses transmitted light microscopy to image the embryos, examine their morphology, and apply selection criteria. Such techniques, however, are subjective, and have produced relatively low success rates (˜10-35%). There is a strong demand for more objective and quantitative assessment methods to increase the accuracy of selection. Time-lapse imaging systems also do not often show clinical efficacy.

Application of artificial intelligence (AI) to morphology data to make the morphology data more quantitative has shown some improvement to embryo selection processes. However, morphology data is a fundamentally limited data set that does not take into account direct biochemistry measurements. Embryos can also be selected for transfer based on data obtained at the genomic, transcriptomic, proteomic and/or metabolomic levels. However, these measurements cannot always be made directly on the embryo without invasive biopsy of cells, which may damage the embryo.

Metabolism has been shown to be an important factor associated with egg and embryo viability. Hence, previous attempts have been made to sample metabolic function as a proxy for viability, including methods of measuring changes in metabolites in embryo culture media (the artificial environment/solution in which the embryo is being grown). Such methods are indirect, however, and attempts to apply them as clinical tools have not been successful. (See, e.g., Vergouw C G, et al., “Day 3 embryo selection by metabolomic profiling of culture medium with near-infrared spectroscopy as an adjunct to morphology: a randomized controlled trial,” Hum. Reprod. August 2012, 27(8), pp. 2304-11). Conversely, autofluorescence can serve as a direct, non-invasive and objective viability prediction tool for eggs and embryos. Autofluorescence directly reflects the biochemistry and metabolism of the sample, and artificial intelligence can be used to convert complex autofluorescence signatures into quantitative probabilistic estimates of viability.

Some studies of autofluorescence imaging have shown value in monitoring Nicotinamide adenine dinucleotide (NADH) and flavin adenine dinucleotide (FAD) using fluorescence lifetime imaging microscopy (FLIM). FLIM can sensitively detect changes or deficiencies in metabolic state or other changes in biochemistry. (See, e.g., Sanchez et al., “Metabolic Imaging with the Use of Fluorescence Lifetime Imaging Microscopy (FLIM) Accurately Detects Mitochondrial Dysfunction in Mouse Oocytes.” Fertil. Steril., December 2018, 110(7), pp. 1387-1397 and Sanchez et al., “Combined Noninvasive Metabolic and Spindle Imaging as Potential Tools for Embryo and Oocyte Assessment,” Hum. Reprod., December 2019, 34(12), Issue 12, pp. 2349-2361). NADH and FAD are both naturally fluorescent chromophores (fluorophores) that are central to metabolism. Hence, NADH and FAD can be monitored non-invasively with fluorescence measurements without the need for foreign stains. This non-invasive monitoring of NADH and FAD is important for IVF, as the introduction of foreign substances would generally not be permitted due to concern over damaging the embryo or egg. The state of NADH and FAD can be determined by the metabolic state of the embryo.

FLIM is an advanced fluorescence technique that can yield much more information about the state fluorophores than standard fluorescence microscopy, which only measures the overall brightness (i.e., the number of photons). FLIM measures the precise arrival time of each fluorescence photon, constructing histograms that reveal the rate of fluorescence decay from the fluorophore's excited state. The shape of the decay curve encodes information about the microenvironment of the fluorophore. This information can be extracted with analysis and artificial intelligence techniques. As such, FLIM can be used to measure one or more quantitative fingerprints of NADH and FAD fluorescence, which sensitively reflect changes or deficiencies in egg or embryo metabolic state. Embodiments described herein relate to the collection, organization, and processing of FLIM data for use in high-accuracy embryo viability determinations and predictions.

In some embodiments, FLIM images can be acquired of 3 z-planes for both NADH and FAD. For each FLIM image, ML can be used to segment and identify the intracellular region. In some embodiments, the intracellular regions of all 3 z-planes, for NADH and FAD can be separately binned, into one aggregate histogram. In some embodiments, the NADH and FAD histograms can be fit with 3-exponential models to obtain 5 fit parameters for each (two species fractions and three fluorescence lifetimes). Also, total photon counts can be used to calculate the fluorescence intensity of the sample, providing a total of 12 quantitative parameters to characterize a metabolic state of the FLIM images. These parameters can be used to train an artificial intelligence algorithm. This algorithm can be used to predict viability of future embryos, measured and analyzed in the same way. In some embodiments, validation methods can be used to validate the model (e.g., k-fold cross validation).

Embodiments described herein can be used for applications beyond embryo viability prediction. For example, FLIM and/or AI methods described herein can be used to assess the efficacy of preparation media for embryos, oocytes, and sperm (e.g., preparation media that is engineered to metabolically enhance gamete function. In some such embodiments, FLIM and/or AI can be used to evaluate the performance of such preparation media.

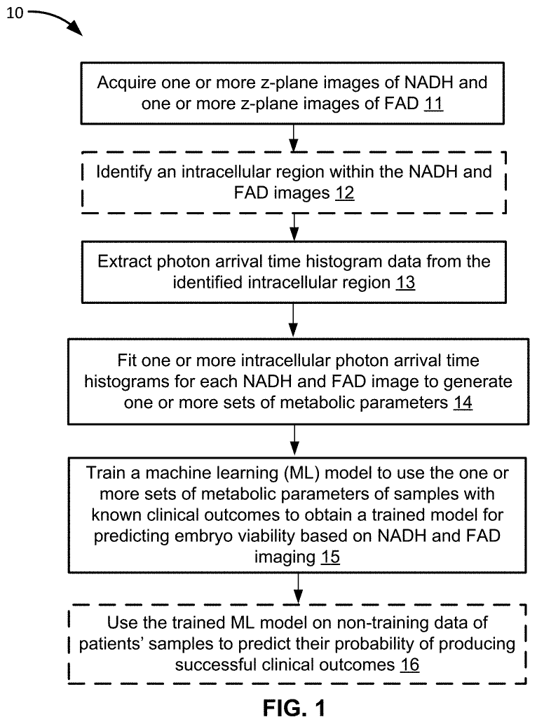

FIG. 1 is a block diagram of a method 10 of predicting embryo viability, according to an embodiment. As shown, the method 10 includes acquiring one or more z-plane (focal plane) images of NADH and one or more z-plane images of FAD at step 11. The method 10 optionally includes identifying an intracellular region within the NADH and FAD images at step 12 (e.g., identifying intracellular regions for each of the three NADH and FAD images). The method 10 then includes extracting photon arrival time histogram data from the images (e.g., from the identified intracellular region) at step 13, and fitting (e.g., to one or more multi-exponential functions) one or more intracellular photon arrival time histograms for each NADH and FAD image to generate one or more sets of metabolic parameters at 14. The one or more sets of metabolic parameters may be referred to as a metabolic fingerprint. In some implementations, the NADH and FAD arrival time histograms may be separately fit, each to a different associated multi-exponential function that includes metabolic parameters forming an associated metabolic fingerprint or portion thereof. The method 10 also includes training a machine learning model, at 15, to use the one or more sets of metabolic parameters of samples with known clinical outcomes to obtain a trained model for predicting embryo viability based on the NADH and FAD imaging at step 11, and optionally includes using the trained model on non-training data of patients' samples to predict their probability of producing successful clinical outcomes at step 16. combining intracellular arrival time histograms for the NADH images at step 14, combining the intracellular arrival time histograms for the FAD images at step 15, and

The method 10 includes acquiring one or more z-plane images of NADH and one or more z-plane images of FAD at step 11. In some embodiments, step 11 includes acquiring 1, 2, 3, 4, 5, 6, 7, 8, 9, 10, or more than about 10 z-plane images of NADH, inclusive of all values and ranges therebetween. In some embodiments, step 11 includes acquiring 1, 2, 3, 4, 5, 6, 7, 8, 9, 10 or more than about 10 z-plane images of FAD, inclusive of all values and ranges therebetween. The NADH and FAD z-plane images form a 3D data structure. In some embodiments, the NADH images can be acquired first, and then the FAD images can be acquired. In some embodiments, FAD images can be acquired first, and then NADH images can be acquired. In some embodiments, NADH images and FADH images can include x and y values that represent pixels, with z values representing fluorescence photon arrival times. In some embodiments, the pixels can include intracellular pixels and extracellular pixels. In some embodiments, the intracellular region can be spatially resolved such that different areas of the biological sample can be separately sampled. In some embodiments, step 11 can include spindle imaging.

In some embodiments, the images can be of an embryo. In some embodiments, the images can be of a gamete. In some embodiments, the images can be of an oocyte. In some embodiments, the images can be of a sperm. In some embodiments, each of the images acquired can be a FLIM image. In some embodiments, the images can include raw intracellular FLIM images. In some embodiments, the images can include normalized intracellular FLIM histograms. In some embodiments, the FLIM can be absent an emission bandpass filter. In some embodiments, the FLIM data can have multiple excitation wavelengths in succession, to obtain a hyperspectral representation of autofluorescence associated with the biological material. In some embodiments, the FLIM data can have a wavelength-splitting optic and a spectrographic detector to obtain a multi spectral representation of the autofluorescence associated with the biological material.

In some embodiments, step 11 can include comparing FLIM data from a plurality sperm cells and selecting a viable sperm cell from the plurality of sperm cells. In some embodiments, step 11 can include analyzing the FLIM data of sperm cells to determine a patent's overall sperm health.

In some embodiments, a single fluorescence image can be acquired with a specified illumination wavelength or collection of wavelengths to excite an endogenous fluorophore (e.g. NADH or FAD) or collection of fluorophores (e.g. NADH and FAD). An intracellular region can then be identified based on NADH and/or FAD data. Intensity values for standard non-FLIM fluorescence can be analyzed with a trained AI algorithm to predict viability. In some embodiments, FLIM can be used instead of a standard fluorescence method. In some embodiments, a specified illumination wavelength and emission filter can be used to image NADH and/or FAD specifically. In some embodiments, additional fluorophores (i.e., in addition to NADH and FAD) can be measured to determine embryo viability. In some embodiments, multiple z-planes can be analyzed for each channel to get better spatial sampling of embryo metabolism. In some embodiments, measurements can be executed during multiple time points or stages of embryo development (e.g., cleavage stage (<8 cells), morula stage, day 5 blast, day 6 blast).

In some embodiments, measurements can be taken with additional imaging modalities. In some embodiments, additional imaging modalities can include a morphology image (e.g., with Brightfield or Hoffman), a second harmonic to image the spindle, and/or a third harmonic to image membranes. In some embodiments, different image processing techniques can be employed. For example, image processing can be employed to correct for non-uniform illumination profiles, as FLIM images are often brighter in the middle than the edges. Processing can include segmenting images to identify intracellular regions or subcellular regions of interest (e.g., the inner cell mass vs. trophectoderm). Processing can include segmenting higher harmonic images to identify the spindle and membrane. Processing can include normalizing pixel values when helpful with AI classifiers.

The method 10 optionally includes identifying an intracellular region within the NADH and FAD images at step 12, the NADH and FAD images acquired in step 11. In some embodiments, identification of the intracellular region can be based on average photon arrival time in the histogram. In some embodiments, the identification of the intracellular region can be based on visual observation.

The method 10 further includes extracting photon arrival time histogram data from the identified intracellular region at step 13. In some embodiments, the extraction of photon arrival time data can be manual. In some embodiments, the extraction of photon arrival time data can be automatic.

In some embodiments, the method 10 can include further processing to analyze FLIM curves. In analyzing FLIM curves, each pixel has a histogram.

The method 10 optionally further includes combining intracellular arrival time histograms for the NADH images. Histograms can be fit (e.g., to a multiexponential fit) to obtain fit parameters that can be fed into artificial intelligence (AI) or machine learning (ML) algorithms. In some embodiments, histograms can also be Fourier-transformed via the Phasor method, and the transformed histograms can be fed into AI or ML algorithms. In some embodiments, histograms from regions can be binned together into aggregate histograms, then fit or Phasor-analyzed. This can increase the signal-to-noise ratio. In some embodiments, rather than fitting, the raw FLIM histograms can be fed directly into an AI algorithm. In some embodiments, FLIM histograms can be normalized as a pre-processing step.

In some embodiments, multiple histograms (e.g., 2, 3, 4, 5 histograms) can be combined to obtain averages and better signal to noise ratios. In some embodiments, the combining intracellular arrival time histograms for the NADH images can include combining all or substantially all intracellular pixels for all of the z-plane images, to obtain one large histogram for NADH.

The method 10 optionally further includes combining the intracellular arrival time histograms for the FAD images. In some embodiments, multiple histograms (e.g., 2, 3, 4, 5 histograms) can be combined to obtain averages and better signal to noise ratios. In some embodiments, the combining can include combining all or substantially all intracellular pixels for all of the z-plane images, to obtain one large histogram for FAD.

The method 10 further includes fitting the NADH and FAD arrival time histograms to a multi-exponential function to obtain metabolic parameters, forming a metabolic fingerprint at step 16. In other words, inputs into AI for embryo prediction can include fit parameters from fitting FLIM decays. In some embodiments, the NADH and FAD arrival time histograms can be fit separately. In some embodiments, the fitting can include one curve for an entire embryo. In some embodiments, parameters from many fits of individual pixels or groups of pixels can be used for the fitting. In some embodiments, phasor coordinate values (e.g., obtained from Fourier transforms of FLIM decays) can be incorporated into AI models for embryo viability predictions. In some embodiments, intensity values of pixels and other texture features of collections of pixels (e.g., variance) can be incorporated into AI models for embryo viability predictions. In some embodiments, second harmonic generation (SHG) images of the spindle can be used for AI-based embryo viability predictions. In some embodiments, spindle morphology (i.e., overall shape), spindle internal organization or “polarity” can be revealed by SHG. SHG can generate a signal with microtubules comprising the spindle oriented parallel, but not anti-parallel. In some embodiments, third harmonic generation (THG) images of membranes can be used to predict embryo viability. In some embodiments, morphology images of the embryo and/or egg can be used to predict embryo viability. In some embodiments, genetic information of the embryo and/or egg can be incorporated into the function and used to predict embryo viability.

In some embodiments, the fitting can include consideration of contextual information, such as patient and clinical factors. In some embodiments, information collected can be fed into a top-level AI algorithm to produce a comprehensive prediction of viability for the embryo or egg. In some embodiments, patient information such as age, body mass index (BMI), genomic information, or any other pertinent health factors can be used to predict embryo viability. In some embodiments, clinic information, such as region, protocol factors, choice of culture medium, or any other pertinent clinic information can be used to predict embryo viability.

In some embodiments, the photon arrival time data can fit Equation (1) below.

$$P ( t ) = ( {F}_{1} {e}^{\frac{- t}{{τ}_{1}}} + {F}_{2} {e}^{\frac{- t}{{τ}_{2}}} + ( 1 - {F}_{1} - {F}_{2} ) {e}^{\frac{- t}{{τ}_{3}}} ) ( 1 - B ) + B \qquad ( 1 )$$

Where:

Fₖ is a fraction of exp k

τₖ is lifetime of exp k

t is time

B is a background term (e.g., a term that adjusts for ambient light or other sources of background light such as very long-lifetime autofluorescence (other than NADH or FAD), etc.)

The “outputs” of the fitting to equation (1) can include the values of F₁, F₂, τ₁, τ₂, τ₃, and I (intensity, obtained by summing photons) for each of NADH and FAD, thus yielding 12 total metabolic parameters.

In some embodiments, the equation that fits the photon arrival time data can be a 2 exponential function fit. In some embodiments, the equation that fits the photon arrival time data can be a 3 exponential function fit. In some embodiments, the equation can be a 4 exponential function fit. In some embodiments, the photon arrival time data can be fit with other decay functions. In some embodiments, fitting the equation can include phasor analysis. In some embodiments, the fitting the equation can include taking a Fourier transform of the decay. In some embodiments, fitting the equation can include assuming a multi-exponential model. In some embodiments, fitting the equation can include principal component analysis. In some embodiments, individual pixels (or n×n binned pixels) can be analyzed to get sub-embryo spatial resolution. In some embodiments, any analysis method can be used to fit the photon arrival time data. In some embodiments, pixels can be analyzed convergently to get viability probability. In some embodiments, each pixel is fit, generating a long lifetime for each. In some embodiments, fitting the equation can include plotting a histogram of lifetimes. For example, the width of the histogram can be indicative of embryo viability. In some embodiments, processing the FLIM data can include noise correction.

The method 10 optionally includes training an AI model for predicting embryo viability based on NADH and FAD imaging at step 17. In some embodiments, the model can include an artificial neural network. In some embodiments, the training can use a plurality of fluorescence photon arrival time histograms of the FLIM data set. In some embodiments, the model can be updated based on feedback generated during subsequent simulations. In some embodiments, the AI model can be trained to classify embryos based on their viability using Naïve Bayes, decision tree, random forest, support vector machines, K nearest neighbors, or any other suitable classification algorithm or combinations thereof. In some embodiments, the AI model can be trained to classify embryos based on their viability embryo analysis via liner regression, lasso regression, logistic regression, multivariate regression, or any other suitable analysis method, or combinations thereof. In some embodiments, the AI model can be trained to classify embryos based on their viability embryo analysis via K-means clustering, fuzzy c-means algorithm, expectation-maximization (EM) algorithm, a hierarchical clustering algorithm, or any other suitable clustering algorithm or combinations thereof. In some embodiments, the AI model can be trained to classify embryos based on their viability embryo analysis via neural networks, such as convolutional neural networks (CNNs), long short term memory networks (LSTMs), recurrent neural networks (RNNs), generative adversarial networks (GANs), radial basis function networks (RBFNs), multilayer perceptrons (MLPs), self-organizing maps (SOMs), deep belief networks (DBNs), restricted Boltzmann machines (RBMs), autoencoders, or any other suitable neural network analysis, or any combination thereof. In some embodiments, validation techniques can be used to analyze embryos, such as resubstitution, hold-out, K-fold cross-validation, LOOCV, random subsampling, bootstrapping, or any other suitable validation technique or combinations thereof.

In some embodiments, training the AI model to use the metabolic fingerprint to obtain a trained model for predicting embryo viability can include a signal filtering technique, supervised artificial intelligence, or unsupervised artificial intelligence prior to estimating likelihood of viable embryo. In some embodiments, estimating the likelihood of a viable embryo can include estimating the likelihood of a successful pregnancy and/or a live birth. In some embodiments, the AI model can be updated based on feedback generated during subsequent estimations.

Embryo metabolism, particularly mitochondrial metabolism, is important and highly associated with embryo viability. Previous attempts to assess mitochondrial integrity as a proxy for embryo viability have been unsuccessful. These methods have included the use of mitochondrial DNA measurements. (Victor A R et al. Accurate quantitation of mitochondrial DNA reveals uniform levels in human blastocysts irrespective of ploidy, age, or implantation potential. Fertil Steril 2017; 107:34-42). Such methods have also included analysis of metabolites in a spent medium, that have failed in a clinic due to high instrument variation. (Vergouw C G et al. Day 3 embryo selection by metabolomic profiling of culture medium with near-infrared spectroscopy as an adjunct to morphology: a randomized controlled trial. Hum Reprod 2012; 27:2304-11).

NADH and FAD are molecules in cells that are central to metabolic and mitochondrial function. They are both naturally fluorescent and can be monitored non-invasively without the need for foreign stains. Their metabolic state and fluorescent signatures can reflect their metabolic state. Cellular species have other sources of autofluorescence that may also reflect biochemistry relevant to viability. (Sutton-McDowall et al., Hyperspectral Microscopy Can Detect Metabolic Heterogeneity within Bovine Post-Compaction Embryos Incubated under Two Oxygen Concentrations (7% versus 20%). Hum Reprod. 2017 Oct. 1; 32(10):2016-2025).

FLIM is an advanced, powerful method of fluorescence microscopy, measuring the microenvironment of fluorophores. It is capable of probing detecting changes or deficiencies in metabolic state or other changes in biochemistry. (Sanchez et al., “Metabolic Imaging with the Use of Fluorescence Lifetime Imaging Microscopy (FLIM) Accurately Detects Mitochondrial Dysfunction in Mouse Oocytes.” Fertil Steril, 2018 December; 110(7):1387-1397). Sanchez et al., “Combined Noninvasive Metabolic and Spindle Imaging as Potential Tools for Embryo and Oocyte Assessment.” Human Reproduction, Volume 34, Issue 12, December 2019, Pages 2349-2361. AI is a powerful tool that can contribute positively to predicting embryo viability. AI has previously been applied to morphological data. (Khosravi, P., Kazemi, E., Zhan, Q. et al. Deep learning enables robust assessment and selection of human blastocysts after in vitro fertilization. npj Digit. Med. 2, 21 (2019)).

FLIM raw data is complex. Even if processed and fit to models, a FLIM measurement outputs multiparametric data sets that do not relate to egg or embryo viability in any obvious way. AI can be applied to FLIM data, or FLIM data combined with other relevant embryo or contextual data to generate more precise predictions of quality than traditional methods of morphology-only assessments.

In some embodiments, the method 10 can include using the trained model on non-training data of patients' samples to predict their probability of producing successful clinical outcomes at step 18. In some embodiments, the non-training data can come from patents. In some embodiments, the non-training data can come from prospective patients. In some embodiments, a successful clinical outcome can refer to a successful pregnancy. In other embodiments, a successful clinical outcome can refer to a live birth. In still other embodiments, a clinical outcome can refer to a biochemical pregnancy, defined as a positive beta-hCG detected between 2-4 weeks of pregnancy. In still other embodiments, a clinical outcome can refer to a miscarriage or a lack of miscarriage.

Although shown and described, with reference to FIG. 1, as being based on photon arrival time histogram data, other methods contemplated by the instant disclosure can be similar to the method of FIG. 1, but based on raw photon arrival time data, rather than photon arrival time histogram data specifically.

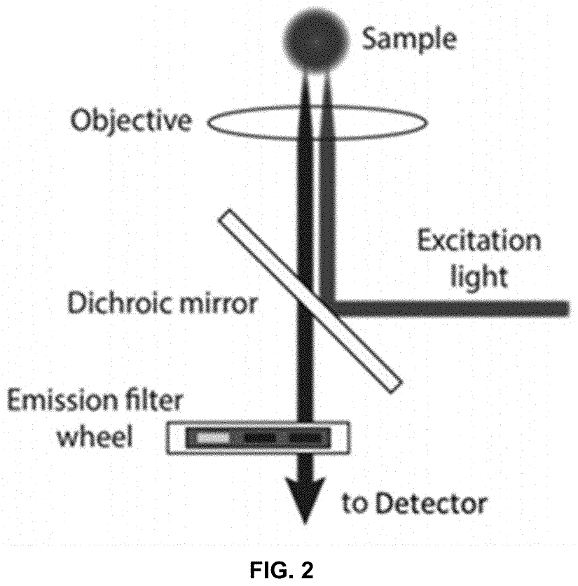

FIG. 2 demonstrates how fluorescence works in the context of fluorescence microscopy. Light of a specific wavelength is used to excite specific fluorophores in a sample to a higher energy state. When the fluorophores de-excite, the fluorophores emit protons of a different color, which can be isolated by wavelength-filtering components. A dichroic mirror and a bandpass filter are often used to isolate these emitted photons in front of a detector. Fluorescence microscopy exists in several methods, including confocal microscopy and multi-photon fluorescence (Sanchez, T., Zhang, M., Needleman, D. & Seli, E. Metabolic imaging via fluorescence lifetime imaging microscopy for egg and embryo assessment. Fertil. Steril. 111, 212-218 (2019)).

Confocal microscopy, or “epi-fluorescence” includes illumination of an extended volume above and below a focal plane, causing out-of-focus fluorescence light to reach the detector and degrade image quality. Confocal microscopy blocks out this out-of-focus light to allow for higher resolution of features in the imaging plane. This is often accomplished by placing a pinhole in front of the detector.

Multi-photon fluorescence is a non-linear optical effect, in which the fluorophores are excited by simultaneously absorbing two photons of lower energy. This is rather than one photon of a higher energy.

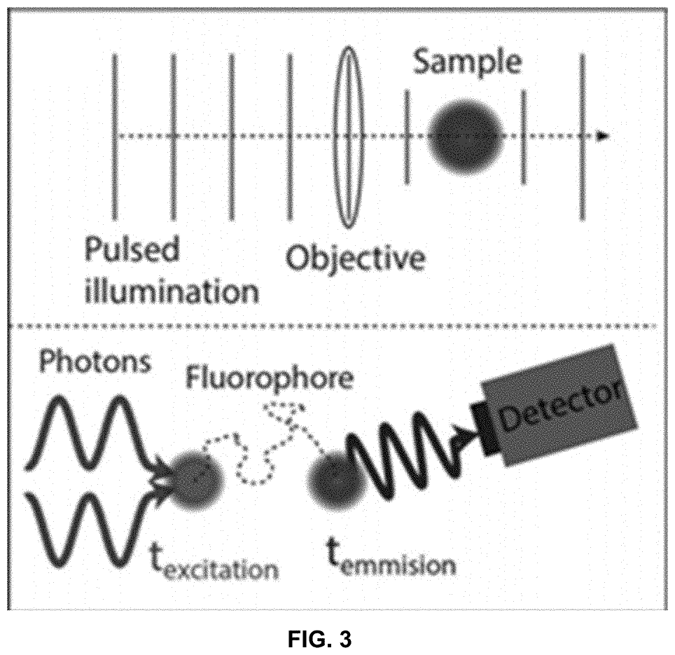

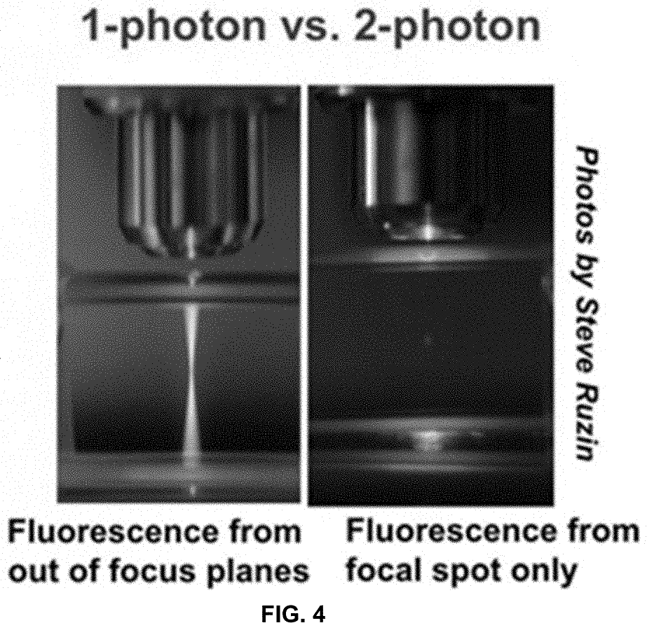

Time-correlated single photon counting (TCSPC) FLIM includes the use of pulsed illumination, as depicted in FIG. 3 Each pulse may excite a single fluorophore. A short time period (i.e., picoseconds to nanoseconds) after the pulse, the molecule's emitted fluorescence photon is detected by a single photon counting detector, and fast-functioning electronics can register a precise arrival time of the fluorescence photon. This significant reduction in excitation is demonstrated in FIG. 4.

Multi-photon absorption is highly proportional to the intensity of illumination. Illumination intensity is brightest in the focal plane. Therefore, excitation is reduced precipitously above and below the focal plane. This provides confocal imaging without needing a pinhole in front of the detector.

Advanced and specialized microscopy forms are also possible, including light-sheet microscopy and super-resolution techniques. Light-sheet microscopy is designed for fast optical sectioning. The configuration involves two objective lenses oriented perpendicular to one another. Super-resolution techniques often include fixing samples. Examples of super-resolution techniques include photoactivated localization microscopy (PALM), stimulated emission depletion (STED), and stochastic optical reconstruction microscopy (STORM).

Several microscopy configurations can be used for FLIM. FLIM techniques can be classified into time-domain and frequency-domain techniques. FLIM techniques can also be classified into photon counting and analog techniques. FLIM techniques can also be classified into point-scanning and wide-field imaging techniques. TCSPC uses time-domain, photon counting, and point-scanning. TCSPC is often used for embryo assessment (Becker, W. Fluorescence lifetime imaging—techniques and applications. J. Microsc. 247, 119-36 (2012)). TSCPC is often used for weakly emitting dyes. NADH and FAD can be considered weakly emitting fluorophores.

Time-domain FLIM includes excitation of a sample with sharp light pulses and measurement of a rate of fluorescence decay as a function of time. This is often on a scale of nanoseconds. Frequency-domain FLIM excites samples with an oscillating intensity light source, and then measures the oscillating fluorescence. This oscillating fluorescence has a phase offset and lower amplitude than excitation light.

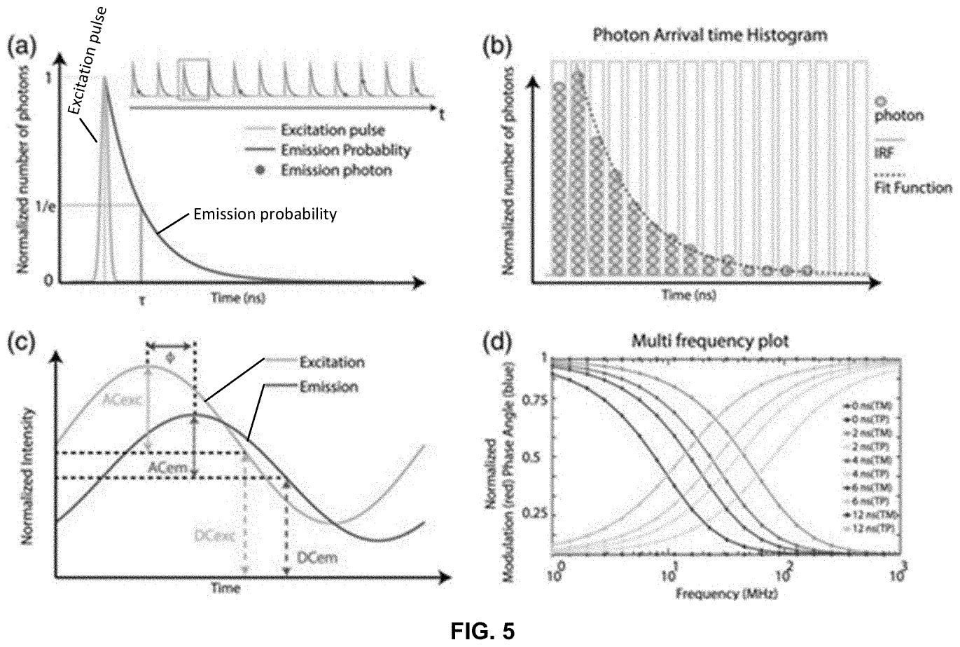

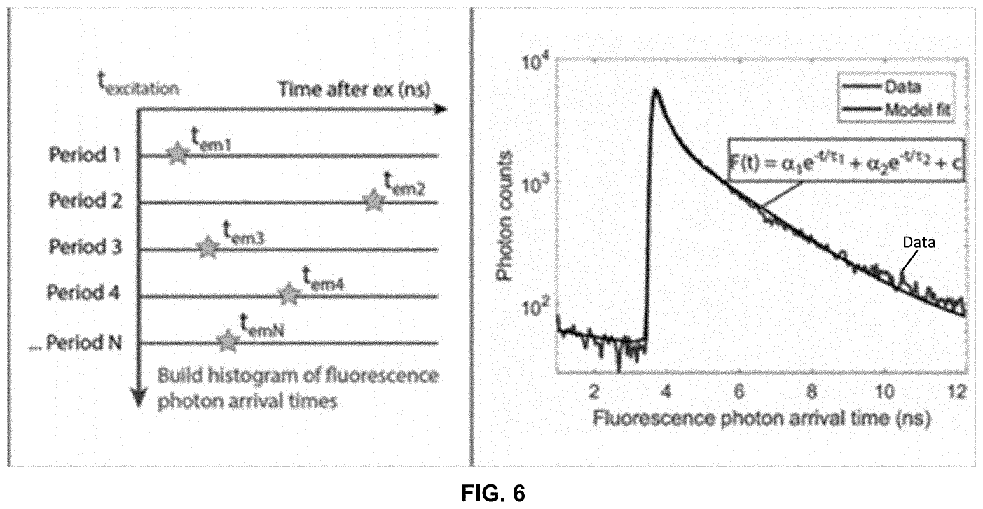

FIG. 5 shows diagrams illustrating time-domain FLIM as well as frequency-domain FLIM (Datta, R., Heaster, T. M., Sharick, J. T., Gillette, A. A. & Skala, M. C. Fluorescence lifetime imaging microscopy: fundamentals and advances in instrumentation, analysis, and applications. J. Biomed. Opt. 25, 1 (2020)). TCSPC is often used for performing time-domain FLIM. TCSPC uses single-photon counting detectors such as photomultiplier tubes (PMT), avalanche photodiodes (APD), or hybrid detectors (e.g., Becker Hickl products) to measure fluorescence emitted from the sample. For each fluorescence photon detected, specialized electronics measure the precise arrival time of the photon (i.e., to the nearest picosecond), constructing histograms of arrival times that represent a microenvironment of a fluorophore. FIG. 6 illustrates this histogram construction (Sanchez, T., Zhang, M., Needleman, D. & Seli, E. Metabolic imaging via fluorescence lifetime imaging microscopy for egg and embryo assessment. Fertil. Steril. 111, 212-218 (2019)). The chart on the left of FIG. 6 demonstrates FLIM delivering pulses at 80 MHz, quickly exciting many fluorophores and recording arrival times. These arrival times are collected into arrival time histograms (at each point in space). The chart on the right of FIG. 6 shows histograms reflecting the microenvironment of the fluorophores, and information can be extracted by fitting the fluorescence decays with models, such as the displayed biexponential decay function. This function represents the decays of encaged and unengaged molecules.

TCSPC is often used if high detection efficiency is desired. Because TCSPC includes counting of each individual photon, it has a very high detection efficiency of signal. This is very important for embryo assessments because it allows for collecting sufficient signal with a minimum of sample illumination. This aids in minimizing risk of photodamage to the embryos. Other methods throw away large portions of the fluorescence, which may require more illumination of the embryo to get the same signal. NADH and FAD have a relatively low quantum yield (ratio of the number of photons emitted to the number of photons absorbed), only about 3-5%. NADH and FAD are weakly emitting fluorophores, so it is desirable to capture as much of their fluorescence as possible. Single photon counting detectors for TCSPC are often very sensitive, such that they are susceptible to background light contamination. Hence, light-tight components or an imaging system to operate in an illuminated clinic are a desired improvement for such an application.

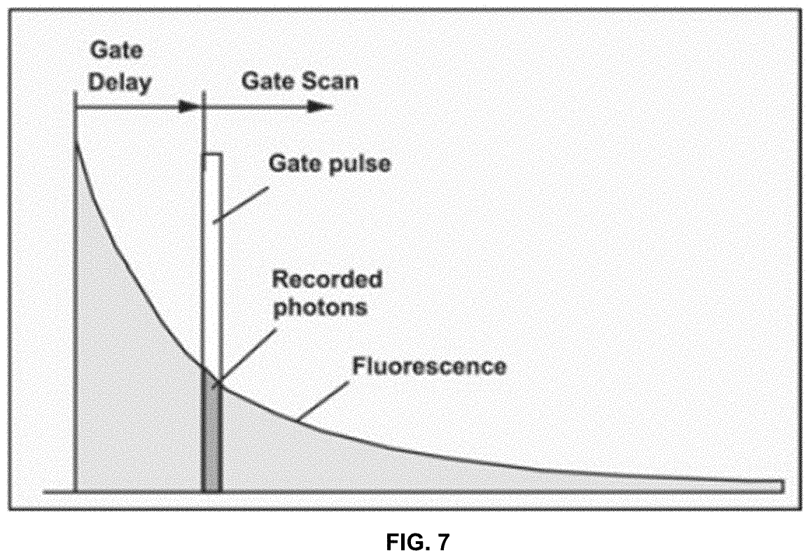

Similar to TCSPC, time-gating FLIM also illuminates the sample with pulsed light. Time-gating FLIM is demonstrated in FIG. 7. After each pulse, camera detectors sample two or more discrete time windows by using gated gain. This technique often has faster acquisition times than TCSPC, but can have several drawbacks. Particularly, most of the fluorescence signal is thrown away. Also, time-gating FLIM can give poor resolution of lifetimes due to undersampling the delay curve. Time-gating FLIM can also lead to poor performance with low photon counts (i.e., weakly emitting fluorophores).

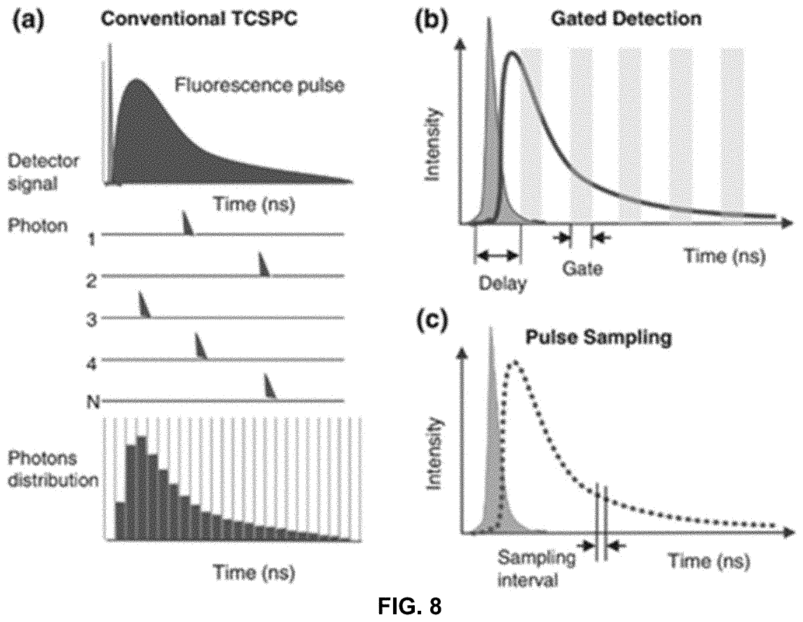

Similar to TCSCP, pulse sampling illuminates samples with laser pulses and measures fluorescence decay. However, while TCSCP measures one photon at a time and builds a histogram, pulse sampling uses a much higher intensity of illumination to excite many fluorophores at once, then uses a fast detector to continuously measure the aggregate fluorescence intensity as the fluorescence decays. FIG. 8 shows plots articulating differences between TCSPC, time-gating, and pulse sampling (Marcu, L. Fluorescence lifetime techniques in medical applications. Ann. Biomed. Eng. 40, 304-31 (2012)).

Several methods can be employed to reduce or substantially eliminate the effect of background light on a fluorescence signal. Laser illumination light can be coupled to an optical fiber, and the optical fiber can be directly pressed against the tissue being probed. This effectively blocks out room light. Detectors can be gated to block background light occurring between laser pulses. TCSPC can achieve a similar result. Using a high intensity signal to illuminate samples can also reduce the relative effects of background light. However, in the field of fertility, coupling of laser illumination light to optical fiber, gated detectors, and high intensity signals can be inappropriate. These techniques often employ a high signal to noise ratio and high illumination. The samples would need to be robust and ample (e.g., tissues or tumors). These techniques can cause damage to an embryo, which is small, delicate, and yields miniscule fluorescence. The use of fiber optics is often spectrographic and does not produce images. Images are valuable for embryo assessment. These techniques can also use single-photon illumination (as multi-photon illumination uses ultrashort pulses, which are more distorted by a fiber optic system). Therefore ultraviolet light would be used, which can be an additional safety concern for the embryo. Additionally, the results of these techniques depend more strongly on instrument features and noise characteristics, introducing uncertainty and variation into the measurements.

Frequency-domain FLIM can include the use of gated gain cameras. Large portions of a fluorescence signal can be thrown away during frequency-domain FLIM. Therefore frequency-domain FLIM has some of the same advantages as time-gating FLIM, but also often performs poorly with low photon counts from weakly emitting fluorophores.

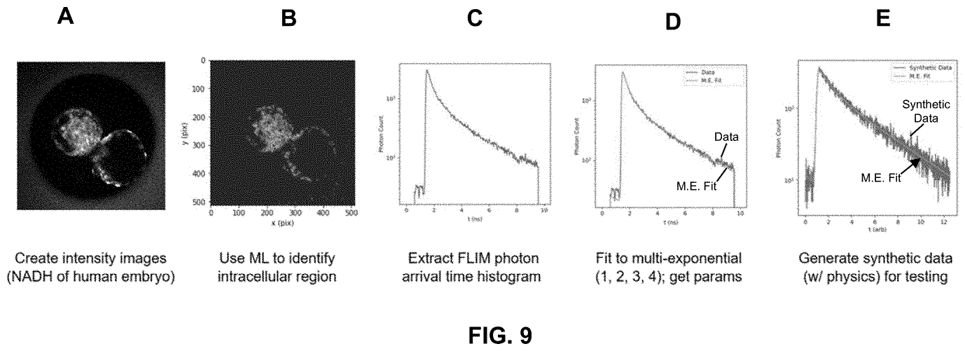

FIG. 9 shows an example of an intracellular region and a series of plots showing a method for embryo viability prediction, in accordance with some embodiments. Plot A shows intensity images created from FLIM data (NADH of human embryo). Plot B shows the intracellular region identified via ML. The brighter, lighter-colored regions are identified as the intracellular region, while the dark background is identified as the extracellular region. Plot C shows a histogram of FLIM photon arrival times retrieved from the FLIM data from within the corresponding intracellular region shown in Plot B. Plot D shows a multi-exponential plot fit to the data from plot C. Plot E shows separate synthetic histogram data, generated for independent testing and algorithm validation, along with a separate multi-exponential fit. Plots D and E are independent data sets.

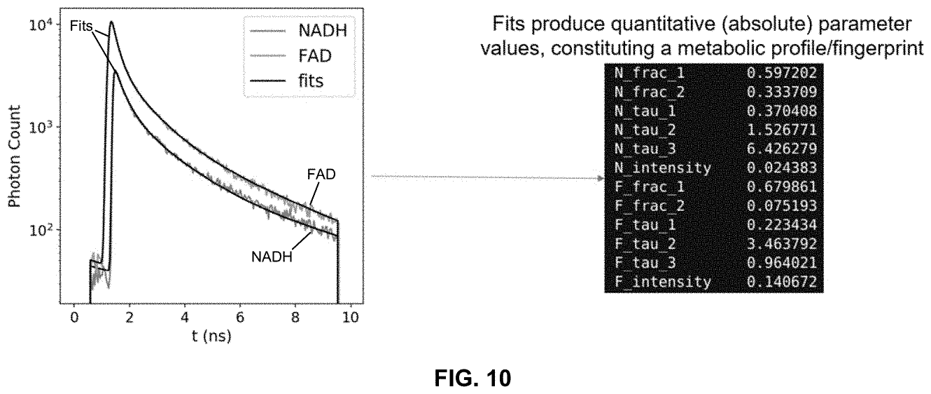

FIG. 10 shows plots of fitted data for human embryo NADH and FAD, and an associated metabolic profile, in accordance with an embodiment. As shown, the plots include a multi-exponential plot fit to photon arrival time data of NADH (lower curve) and a multi-exponential plot fit to photon arrival time data of FAD data (upper curve). The multi-exponential fit for FAD is described by the following equation.

$$P ( t ) = ( 0.68 {e}^{\frac{- t}{0.22}} + 0.075 {e}^{\frac{- t}{3.46}} + ( 1 - 0.68 - 0.075 ) {e}^{\frac{- t}{0.96}} ) \qquad ( 2 )$$

The multi-exponential fit for NADH is described by the following equation.

$$P ( t ) = ( 0.6 {e}^{\frac{- t}{0.37}} + 0.33 {e}^{\frac{- t}{1.53}} + ( 1 - 0.60 - 0.33 ) {e}^{\frac{- t}{6.43}} ) \qquad ( 3 )$$

The multi-exponential fits produce quantitative (absolute) parameter values, constituting a metabolic profile/fingerprint, as shown in the righthand side of FIG. 10. AI can then be used to convert the metabolic profile into (or to generate) a prediction of viability.

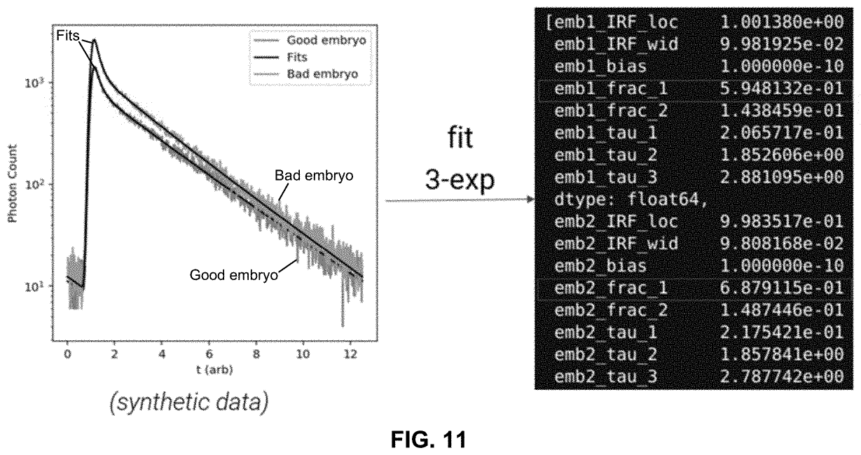

FIG. 11 shows example synthetic NADH fit data for a “good” (i.e., viable) embryo (lower curve) and a “bad” (i.e., not viable) embryo (upper curve). The NADH data shown in FIG. 11 is synthetic data. As shown, the multi-exponential fit for the good embryo is described by the following equation.

$$P ( t ) = ( 0.59 {e}^{\frac{- t}{0.21}} + 0.14 {e}^{\frac{- t}{1.85}} + ( 1 - 0.59 - 0.14 ) {e}^{\frac{- t}{2.88}} ) \qquad ( 4 )$$

The multi-exponential fit for the bad embryo is described by the following equation.

$$P ( t ) = ( 0.69 {e}^{\frac{- t}{0.22}} + 0.15 {e}^{\frac{- t}{1.86}} + ( 1 - 0.69 - 0.15 ) {e}^{\frac{- t}{2.79}} ) \qquad ( 5 )$$

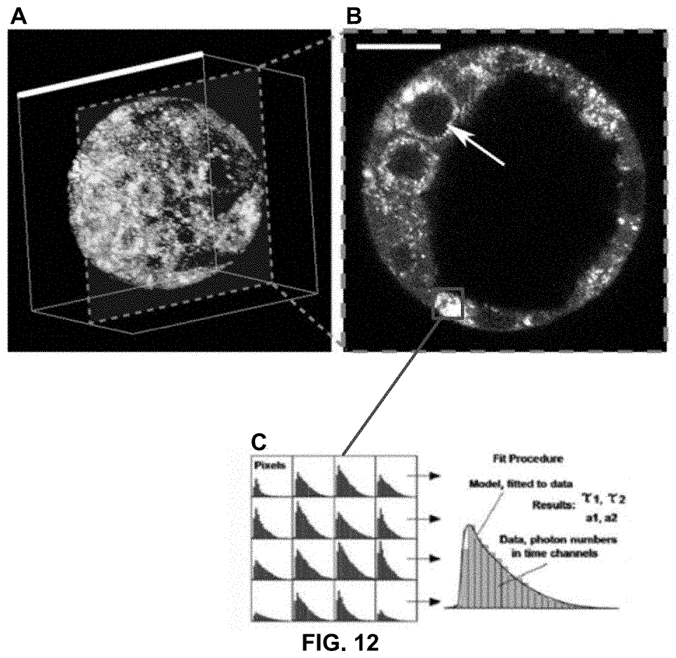

FIG. 12 shows FLIM imagery of a single embryo, in an example embodiment. The data comprising the FLIM images (FLIM data) was collected for 3 z-planes (focal planes), to ensure adequate spatial sampling, and for two metabolic parameters: NADH followed by FAD, so 6 FLIM images in total. Inset A of FIG. 12 shows a 3D reconstruction of the embryo based on the multiple focal planes (with 25-μm scale bar), for NADH, and inset B of FIG. 12 shows an intensity image created from the FLIM data (NADH), facilitating the visualization of subcellular structures, including nuclei (see arrow annotation and 25-μm scale bar). As discussed above, each FLIM image is a three-dimensional (3D) data structure in which the x and y values represent the pixels and z values represent fluorescence photon arrival times (for each pixel). Inset C of FIG. 12 shows histograms of the fluorescence photon arrival times retrieved from the FLIM data of inset B, from within the annotated portion of an intracellular region thereof (indicated by a box). As shown in FIG. 12, the histograms from each of the relevant pixels can be combined to obtain averages and to improve signal to noise ratios. Histograms from all intracellular pixels can be combined, for all 3 z (focal) planes to obtain a single consolidated histogram for each of NADH and FAD.

FIG. 13 shows FLIM data and analysis for a human embryo in an example embodiment. Inset A of FIG. 13 shows an intensity image created from FLIM data (NADH of a human embryo). Inset B of FIG. 13 shows an intracellular region of the intensity image of inset A, as identified using machine learning. Inset C of FIG. 13 shows a histogram of FLIM photon arrival times extracted from the intracellular region data shown in inset B. Inset D of FIG. 13 shows a multi-exponential fit of the data from inset C. In this particular example, inset D shows a fit of the data of inset C to the following 2-exponential equation:

$$P ( t ) = ( {Fe}^{\frac{- t}{{τ}_{1}}} + ( 1 - F ) {e}^{\frac{- t}{{τ}_{2}}} ) ( 1 - B ) + B \qquad ( 6 )$$

The quantitative parameters resulting from the foregoing NADH analysis are as follows:

| | |
|---|---|
| F₁ | 0.70509 |
| τ₁ | 0.45202 |
| τ₂ | 2.44419 |
| I (intensity) | 1.19693 |

A similar procedure (i.e., creating FLIM intensity images, using machines learning to identify an intracellular region, extracting a FLIM photon arrival time histogram, and fitting the arrival time histogram data to a multi-exponential equation) can be performed for FAD, resulting in a total of 8 metabolic parameters (4 metabolic parameters for each of NADH and FAD).

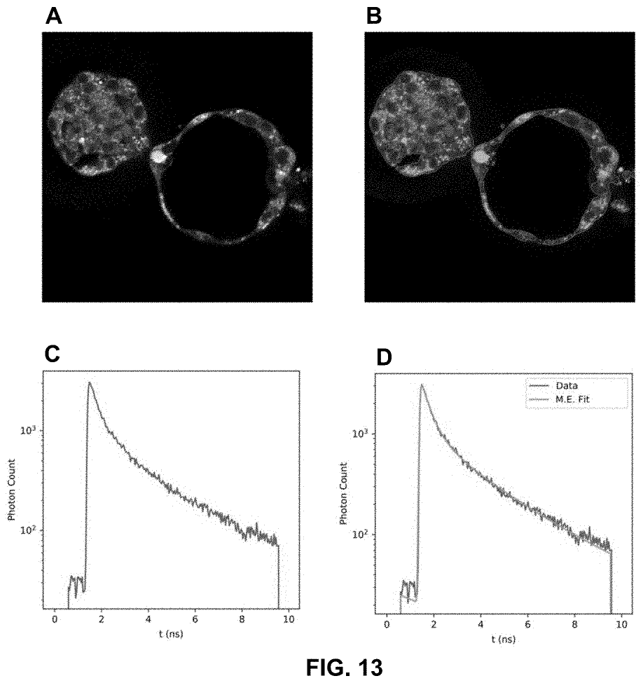

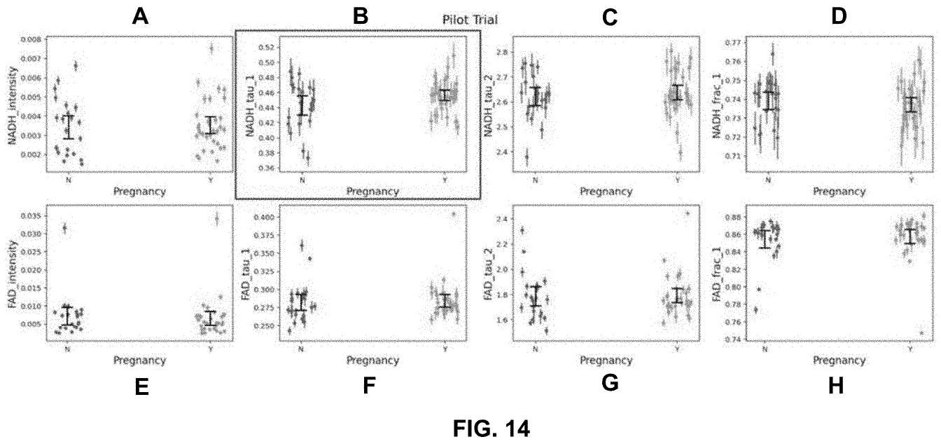

FIG. 14 shows the plots A-H of various parameters obtained from histogram plots of FLIM data for a pilot trial performed for 55 human embryos. The parameters evaluated include F₁, τ₁, τ₂, and intensity I. Plots A-D represent the four parameters associated with NADH, and plots E-H represent the four parameters associated with FAD. The parameter values in each of the plots A-H have been classified for two outcomes of the pilot trial: (a) those embryos that led to a successful pregnancy (denoted by Y on the x-axis) and (b) those embryos that did not lead to a successful pregnancy (denoted by N on the x-axis). Of the 8 parameters shown in plots A-H, the τ₁, parameter (short lifetime) for NADH showed the most statistically significant difference for, or separation between, Y and N outcomes.

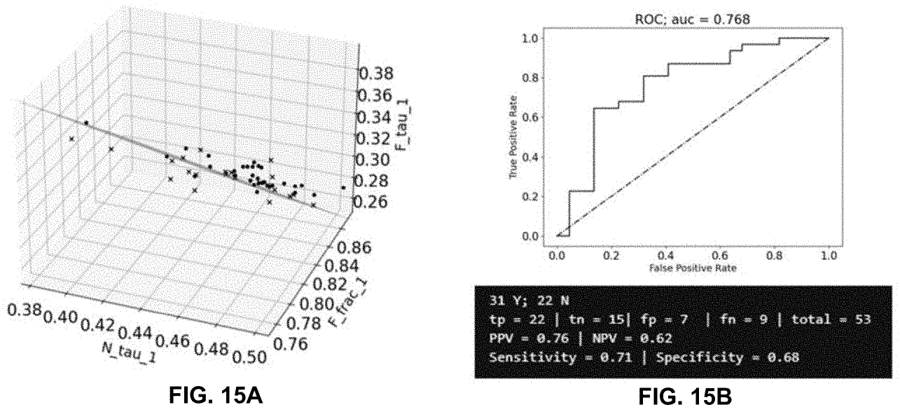

FIGS. 15A-15B show the results of a logistical regression analysis. More specifically, FIG. 15A is a plot showing a logistic regression fit identifying three most significant parameters (NADH τ₁, FAD τ₂, and FAD F₁) for embryo viability prediction, and FIG. 15B is a ROC curve showing true positive rate vs. false positive rate for the data of FIG. 15A. FIG. 15A shows the separating plane from the logistic regression fit, with a p-value of p=0.014 (<0.05), indicating a good fit. In FIG. 15B, the dotted line represents a ratio of 1. The values obtained reside almost exclusively within the True Positive region of the graph, and the associated sensitivity and specificity were 0.78 and 0.63, respectively. FIGS. 15A-15B demonstrate that analyzing metabolic parameters jointly provides better data separation (e.g., better discrimination of samples).

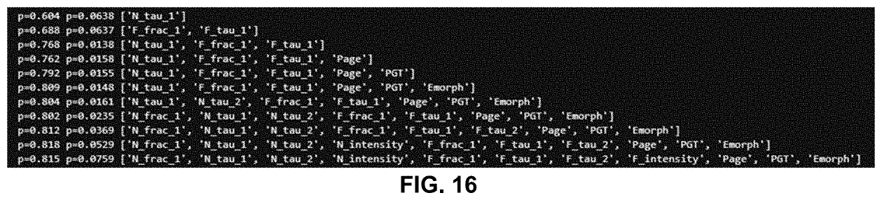

FIG. 16 shows p-values for various multi-parameter models, for the data set of FIGS. 15A-15B. More specifically, the best-separating parameters for each of a 1-parameter model, a 2-parameter model, and so forth, are shown. The parameters evaluated combined included NADH τ₁, NADH τ₂, NADH F₁, NADH intensity, FAD τ₁, FAD τ₂, FAD F₁, FAD intensity, patient age (“page”), morphology (“Emorph”), and PGT testing status (“PGT”). Parameters that are not metabolic parameters may be referred to as “contextual factors.” As can be seen in FIG. 16, NADH τ₁, FAD τ₁, and FAD F₁, were typically found to be included with the best separators, and patient age was found to be the best contextual factor that contributed to a meaningfully lower p-value.

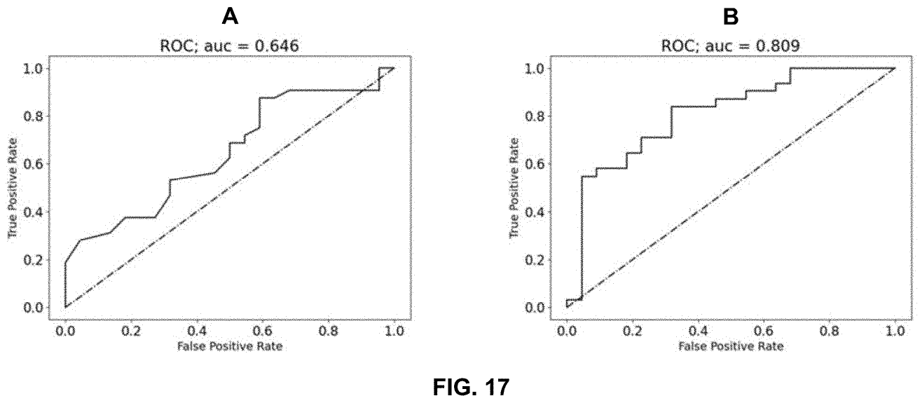

FIG. 17 shows ROC curves comparing true positive rates vs false positive Rates obtained using logistical regression analysis, with differing combinations of parameters used to predict the success rate of IVF. More specifically, FIG. 17 compares ROC curves for (1) viability predictions based on morphology, age, and pre-implantation genetic testing (PGT) status only, and (2) viability predictions based on metabolic parameters combined with the contextual parameters from (1), in accordance with some embodiments. Inset A of FIG. 17 shows the results when considering a combination of morphology, age, and PGT testing status only, with an area under the receiver operating characteristic (ROC curve (“AUC”) of 0.646, as an aggregate measure of performance (or prediction accuracy) for the noted combination. As shown at inset A of FIG. 17, when additional parameters NADH τ₁, FAD τ₁, FAD F₁, and FAD τ₂ were added, the prediction accuracy increased substantially, with an AUC of 0.809.

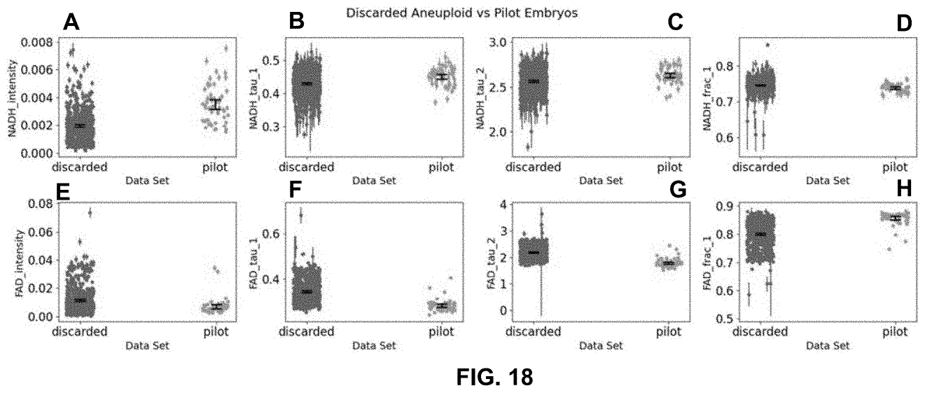

FIG. 18 shows plots A-H of various parameters obtained from histogram plots of FLIM data, comparing embryos that were imaged during the pilot trial with discarded aneuploid embryos (having a significantly larger sample size). This set of aneuploid embryos was PGT tested, which is typically done primarily for morphologically acceptable (i.e., “good” morphology) embryos, but they were discarded because the PGT test deemed they were aneuploid. The metabolic parameters evaluated were F₁, τ₁, τ₂, and intensity. Plots A-D represent the four parameters for NADH, and plots E-H represent the four parameters for FAD. FLIM imaging was done under the same conditions for all evaluated embryos. On almost all the parameters, the pregnant embryos from the pilot trial (a) are statistically different/distinguishable from the discarded aneuploid embryos.

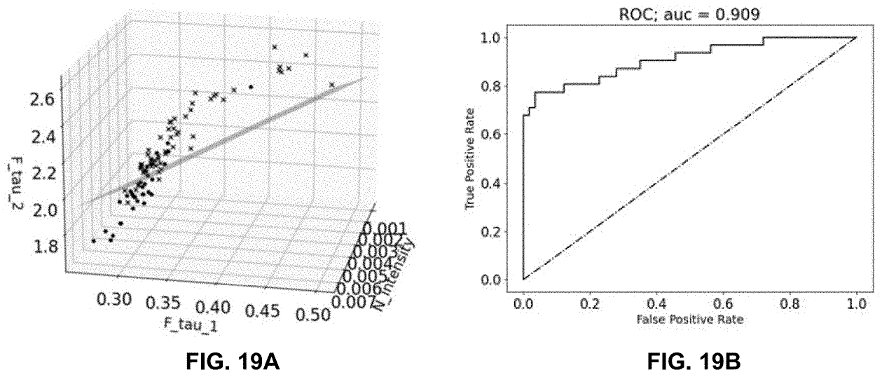

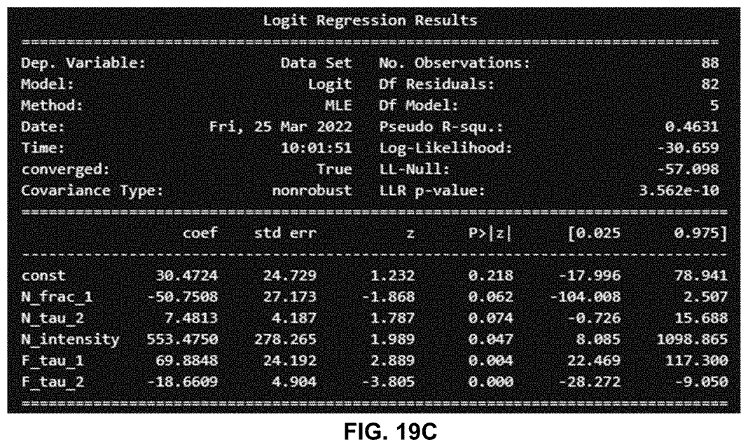

FIGS. 19A-19B show the results of a logistical regression analysis of the pilot-pregnant embryos vs. discarded aneuploid embryos. More specifically, FIG. 19A is a plot showing a logistic regression fit identifying three most significant parameters (FAD τ₁, FAD τ₂, and NADH intensity), and FIG. 19B is a ROC curve showing true positive rate vs. false positive rate for the data of FIG. 19A. In FIG. 19B, the dotted line represents a ratio of 1. The values obtained reside exclusively within the true positive region of the graph, and AUC was 0.909. FIGS. 15A-15B demonstrate that analyzing metabolic parameters jointly provides better data separation (e.g., better discrimination of samples). This analysis shows that the pilot pregnant embryos can be clearly distinguished from discarded aneuploid, using methods of the present disclosure, and demonstrates the efficacy of FLIM data and the accompanying statistical analysis to predict the success for an embryo leading to successful pregnancy. FIG. 19C shows the logistic regression results associated with FIG. 19A. As shown in FIG. 19C, the logistic regression of 5 parameters has a p-value of 4×10⁻¹⁰.

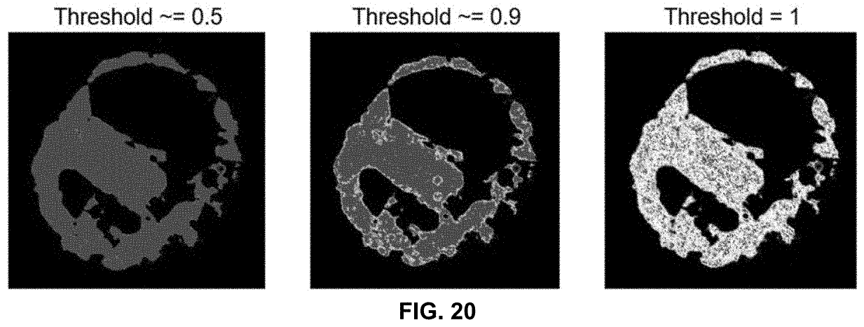

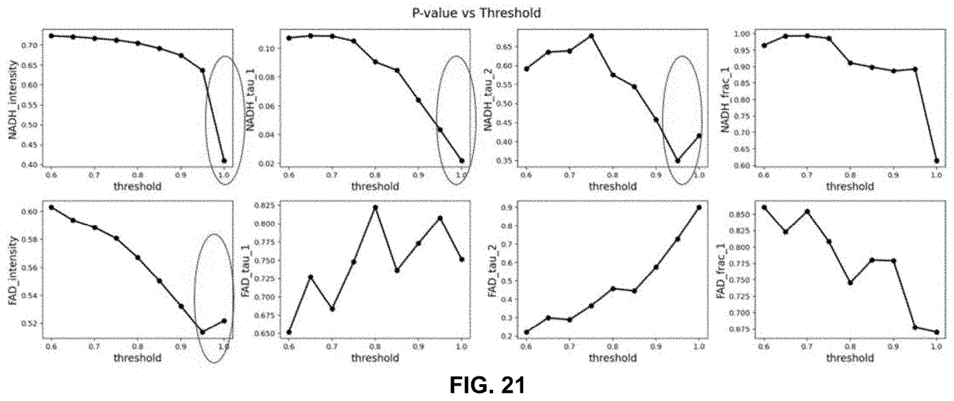

FIG. 20 shows image segmentation probability maps for intracellular pixels of a FLIM-imaged embryo, for various different probability thresholds, in accordance with an embodiment. Intracellular pixels were identified using a machine learning algorithm that generates probabilities. The probability threshold (also called a segmentation threshold) can be changed, to vary which pixels are included in the fit. For example, as shown in FIG. 20, a lower segmentation threshold (˜=0.5, as shown in the leftmost image of FIG. 20) leads to a larger number of pixels being counted as part of the intracellular region, whereas a higher segmentation threshold (=1, as shown in the rightmost image of FIG. 20) leads to a smaller number of pixels (e.g., only the pixels to which the ML algorithm assigned the highest probabilities of being in the “intracellular channel”) being counted as part of the intracellular region. The latter can result in an improvement to the signal to noise ratio of the FLIM data (e.g., because pixels that may be contaminating the signal may be excluded), and/or can result in the best statistical separation. FIG. 21 includes plots of p-value vs. threshold, the plots comparing T-tests of pregnancy vs. non-pregnancy for each metabolic parameter, in accordance with an embodiment. As can be seen FIG. 21, for a subset of the plots, the p-value drops significantly as the segmentation threshold nears the value of 1, indicating a high degree of accuracy in predicting successful pregnancy with the data fitting. Without wishing to be bound by theory, it is hypothesized that with higher segmentation threshold values, a higher proportion of the mitochondrial metabolic activity compared to the cytoplasmic activity is represented in the FLIM data, thereby leading to a higher accuracy in predicting successful pregnancy by the data fit models.

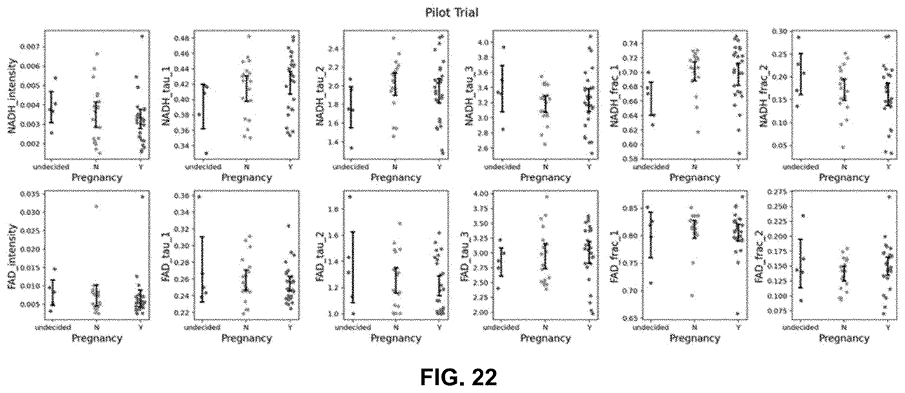

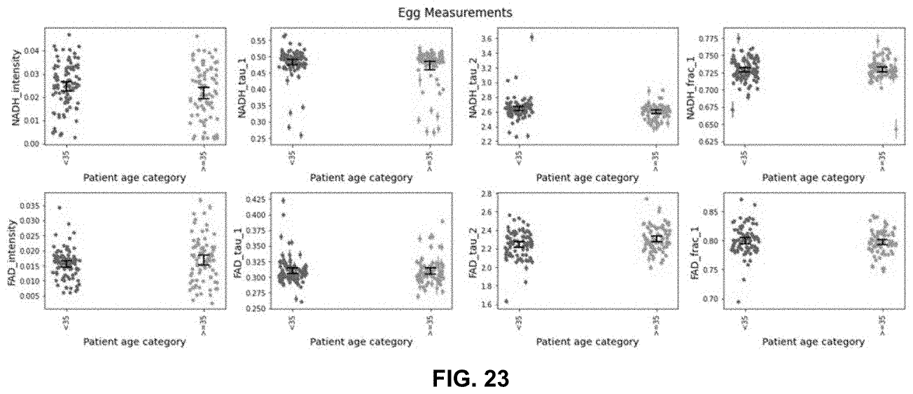

FIG. 22 includes plots of metabolic parameters vs. pregnancy outcomes, plots showing data separation among embryos resulting in pregnancy and embryos not resulting in pregnancy, using a 3-exponential function, in accordance with some embodiments. FIG. 23 includes plots of human discarded oocyte (egg) metabolic parameters vs. patient age categories, in accordance with some embodiments. Data was compiled for >100 discarded oocytes, the data including patient age, egg morphology, and other factors. As can be seen in FIG. 23, age of patient (e.g., as a proxy for egg quality) can be differentiated based on the metabolic parameters. As used herein, the phrase “metabolic parameters” can include intensity.

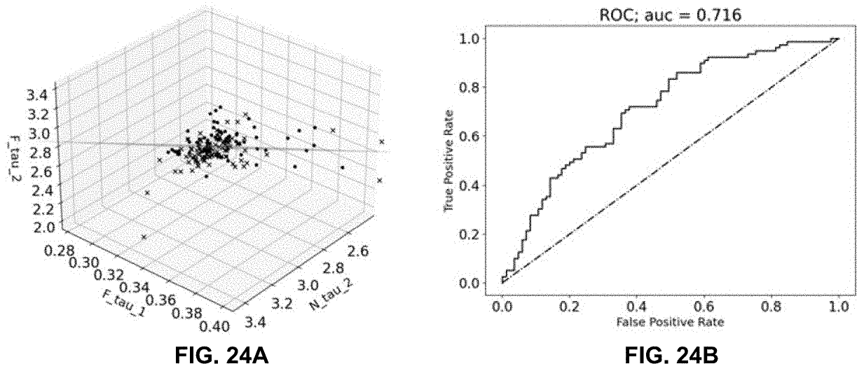

FIGS. 24A-24B show the results of a logistical regression analysis for eggs from older patients vs. younger patients, in accordance with some embodiments. More specifically, FIG. 24A is a plot showing a logistic regression fit identifying three most significant parameters (FAD τ₁, FAD τ₂, and NADH τ₂), and FIG. 24B is a ROC curve showing true positive rate vs. false positive rate for the data of FIG. 24A. FIG. 24A shows the separating plane from the logistic regression fit, and the symbols “⋅” represent patients aged >=35 years old, while the symbols “x” represent patients aged <35 years old. In FIG. 24B, the dotted line represents a ratio of 1. The AUC of the ROC curve is 0.716, and the p-value of the logistic regression is 7×10⁻⁵—indications of the performance of this method to distinguish eggs of older patients vs. younger patients. FIGS. 24A-24B demonstrate that older eggs and younger eggs show significant differences when analyzed according to methods set forth herein.

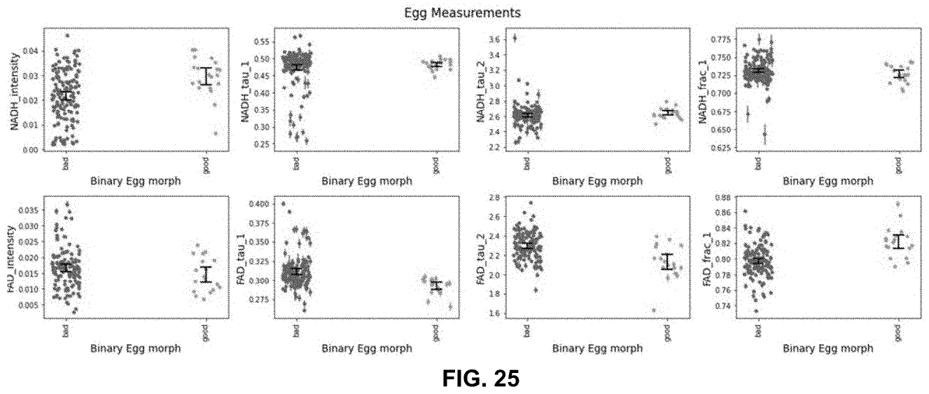

FIG. 25 includes plots of various metabolic parameters, obtained from histogram plots of FLIM data for discarded eggs, showing data separation between discarded eggs having good morphology vs. bac morphology, in accordance with some embodiments. The parameters evaluated were F₁, τ₁, τ₂, and intensity. The upper row of plots represent the four parameters for NADH and the lower row of plots represent the four parameters for FAD. Each plot shows datapoints representing eggs that are classified as having good (desirable) morphology or bad (undesirable) morphology.

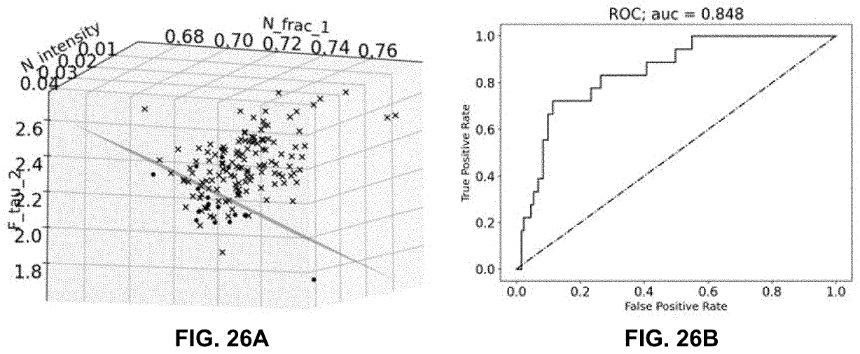

FIG. 26A a plot showing a logistic regression fit for discarded eggs having good vs. bad morphology, identifying three most significant FLIM parameters for differentiating egg morphology—namely, FAD τ₁, NADH τ₁, and NADH intensity. FIG. 26B is a ROC curve showing true positive rate vs. false positive rate for the data of FIG. 26A. In FIG. 26B, the dotted line represents a ratio of 1. The AUC of the ROC is 0.848, and the p-value of the logistic regression is 1×10⁻⁵.

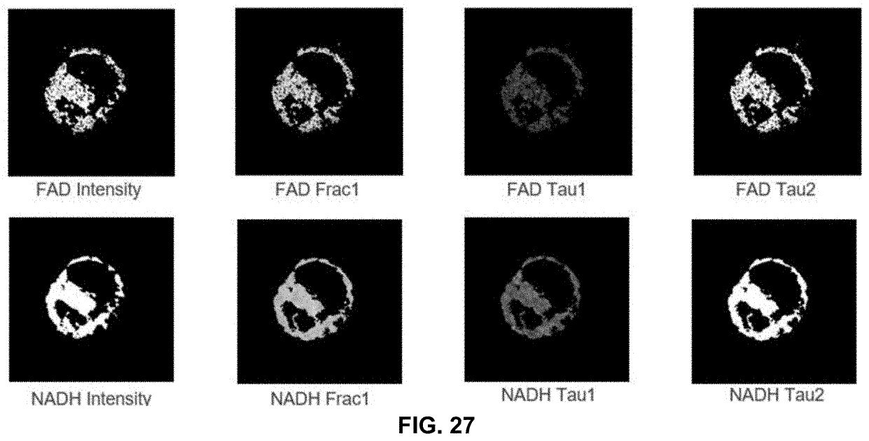

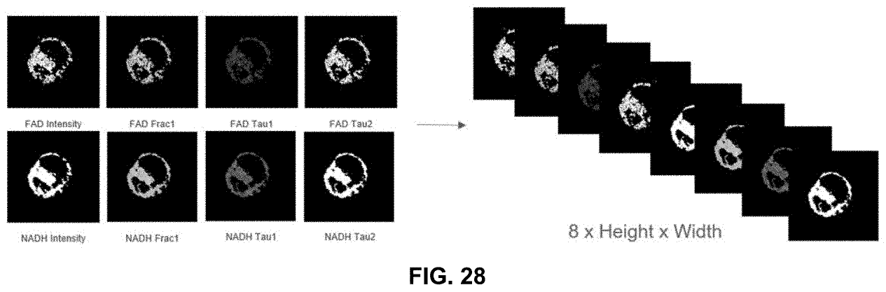

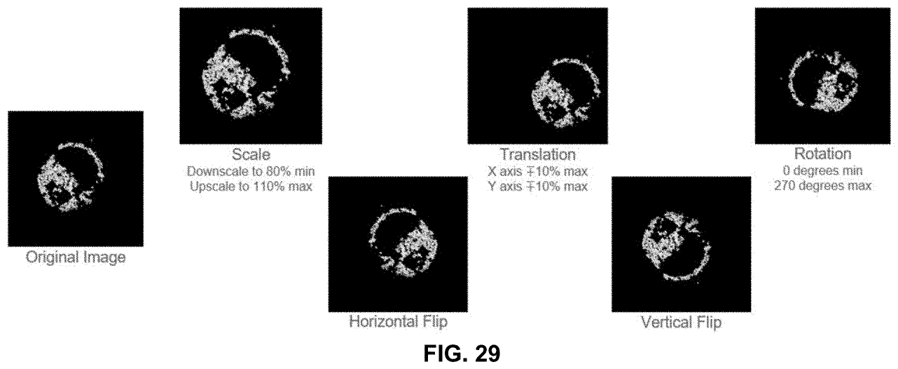

FIG. 27 shows example FLIM metabolic parameter images for each of FAD and NADH, in accordance with some embodiments. The methodology for generating these images included 5-pixel binning to fit all intracellular pixels using a 2-exponential function, resulting in 8 TIFF images (512×512 resolution) for the 8 parameters extracted from the 2-exponential fitting (i.e., F₁, τ₁, τ₂, and intensity for each of FAD and NADH). FIG. 28 shows the channel stacking (i.e., concatenation in the 3^{rd} dimension) of the 8 TIFF images of FIG. 27 to create one composite image that can subsequently be fed into a model (e.g., a neural network (NN) model). In some implementations, prior to feeding the composite image into the model, data augmentation is performed. Depending on the model, any one or combination of the augmentations shown in FIG. 29 can be applied, e.g., pseudo-randomly, to all 8 images. As all 8 images are stacked, a random augmentation operation may be applied to fir all images simultaneously or substantially simultaneously/overlapping in time. As shown in FIG. 29, the leftmost image is an example original image, and example augmentations to scale (e.g., within a range of 80% to 110%), horizontal flip, lateral translation along X and/or Y axes (e.g., within a range of +/−10%), vertical flip, and rotation (e.g., within a range of 0 to 270 degrees) are shown.

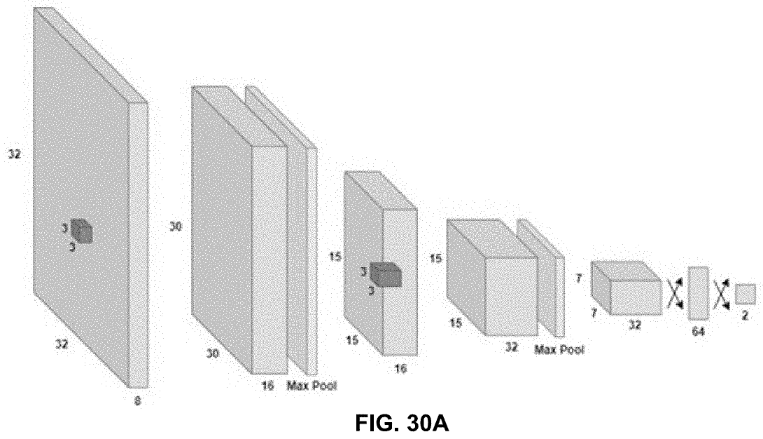

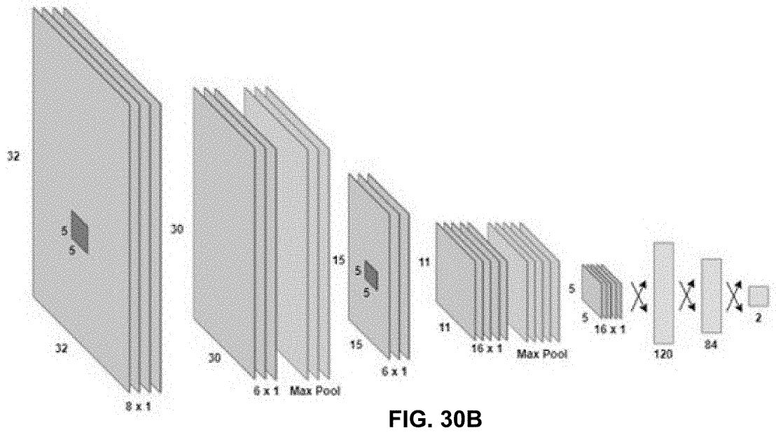

FIG. 30A shows an example architecture of a neural network (NN) model, “EmbryoNet,” that represents fit parameters as channels in a third dimension, in accordance with an embodiment. EmbryoNet is a small NN that represents fit parameters as channels in the third dimension, and is configured to preserve simplicity.

FIG. 30B shows an example architecture of a NN model, “EmbryoNet 3D,” that represents fit parameters as channels in a fourth dimension, in accordance with an embodiment. The EmbryoNet 3D model is a small NN that represents fit parameters as channels in the fourth dimension, and is configured to capture the ‘changes’ with time.

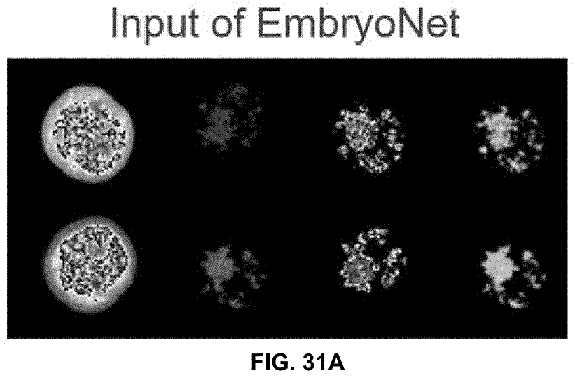

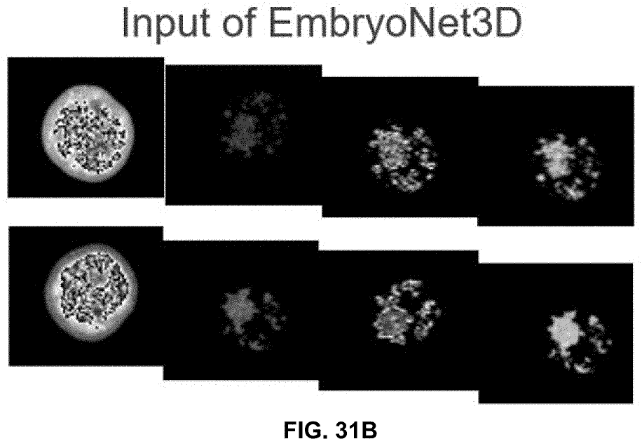

An example data set included (a) pilot data for 43 patients, including 25 pregnant patients and 18 non-pregnant patients, and (b) discarded data for 41 patients including 15 aneuploid and 26 other. Plots A and B show the input images of EmbryoNet and EmbryNet 3D models. Table C shows the results of the data fitting using the EmbryoNet model showing high F1 scores and high accuracy. FIG. 31A is an image of an example input to the EmbryoNet model, in accordance with an embodiment, and FIG. 31B is an image of an example input to the EmbryoNet 3D model, in accordance with an embodiment. The results generated for the data sets using the EmbryoNet model were as follows:

| | # of | F1 | | | |
|---|---|---|---|---|---|
| Dataset Name | Patients | Score | Precision | Recall | Accuracy |
| Pregnant-Non-Pregnant | 43 | 93% | 88% | 100% | 87.5% |
| Pilot-Discarded | 84 | 78% | 78% | 78% | 75% |
| Pregnant-Discarded | 66 | 80% | 80% | 80% | 84.62% |
| Pregnant-Aneuploid | 40 | 80% | 67% | 100% | 75% |

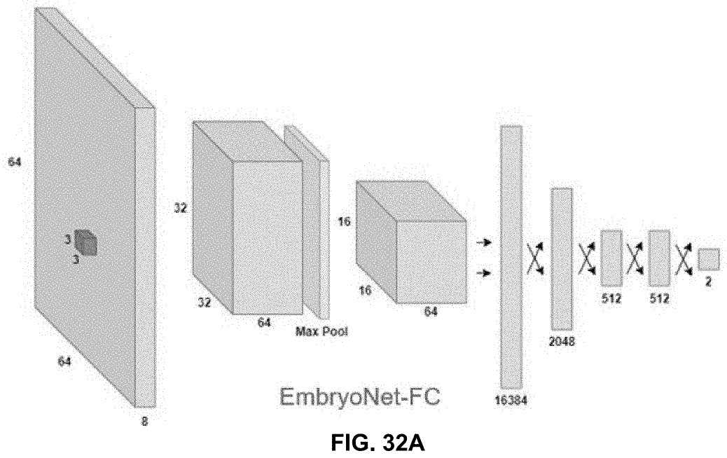

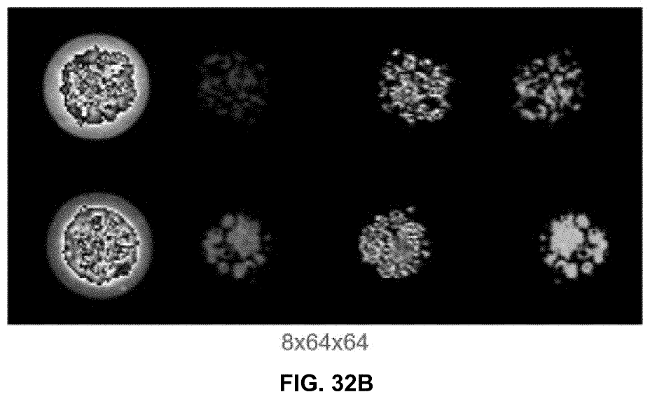

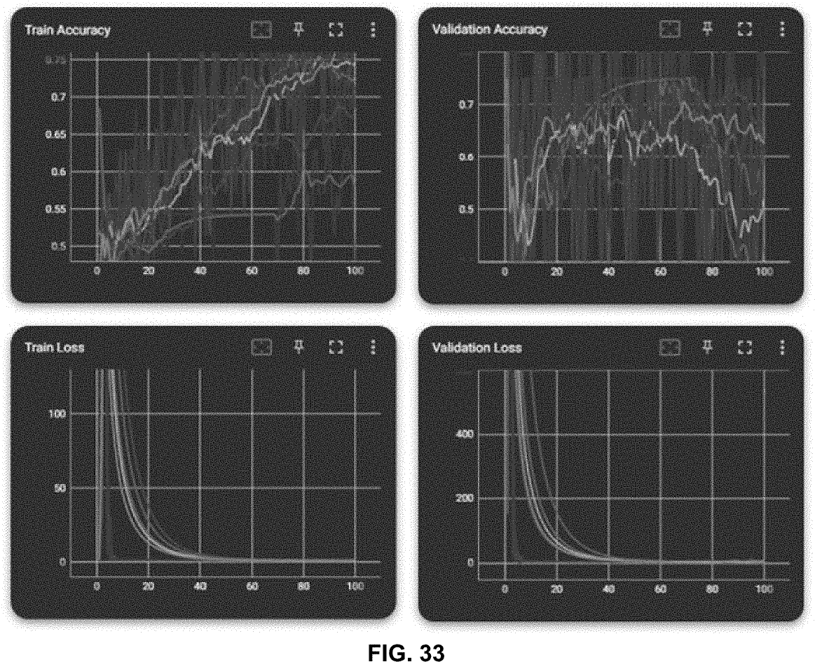

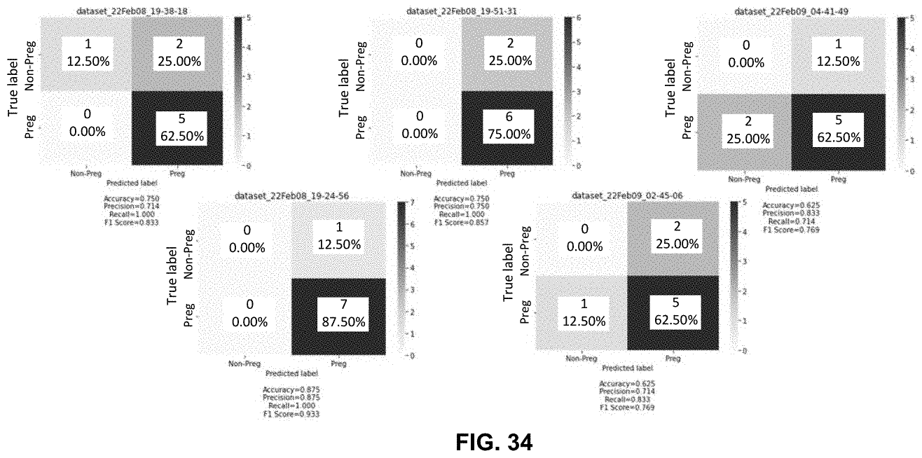

FIG. 32A shows an example architecture of a NN model with a fully connected (FC) head, “EmbryoNet-FC,” in accordance with an embodiment. This model aims to preserve as much information as possible between pixels by having a complex FC head and applying fewer convolutions. FIG. 32B is an image of an example input to the EmbryoNet-FC model, in accordance with an embodiment. FIG. 33 shows training accuracy, validation accuracy, training loss, and validation loss plots for the EmbryoNet-FC model, in accordance with an embodiment. The FC Network is trained at once and the validation accuracy decreased after around 60 epochs due to overfitting. From among the data augmentations shown and described with reference to FIG. 29, flip was the data augmentation used during the EmbryoNet-FC training process. The dataset included 35 training samples and 8 test samples, which included 25 pregnant and 18 non-pregnant samples. The average results were as follows: F1=83.25%, precision=77.74%, recall=90.95%, and accuracy=72.5%. FIG. 34 shows confusion matrices of results of the EmbryoNet-FC model for the pilot dataset, showing high prediction accuracy overlaps between true pregnancy and predicted pregnancy.

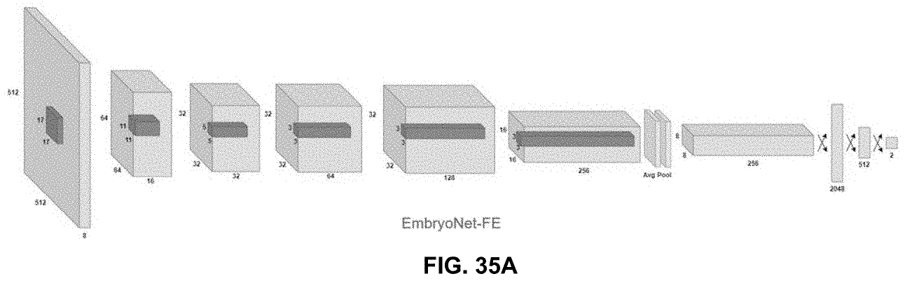

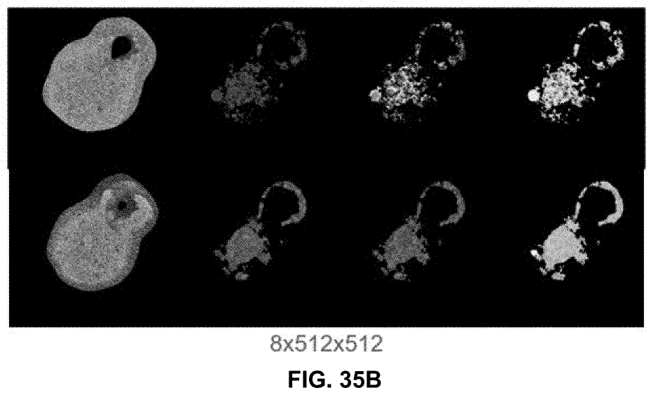

FIG. 35A shows an example architecture for a feature extraction (FE) model based on EmbryoNet (“EmbryoNet-FE”), in accordance with an embodiment. The EmbryoNet-FE model is configured to overfit distribution of the data set using data augmentation. It is observed that many passes of the dataset over the network may be needed to optimize feature extraction, and large kernels may be used for retaining information between further pixels. FIG. 35B is an image of an example input to the FE model of FIG. 35A, in accordance with an embodiment.

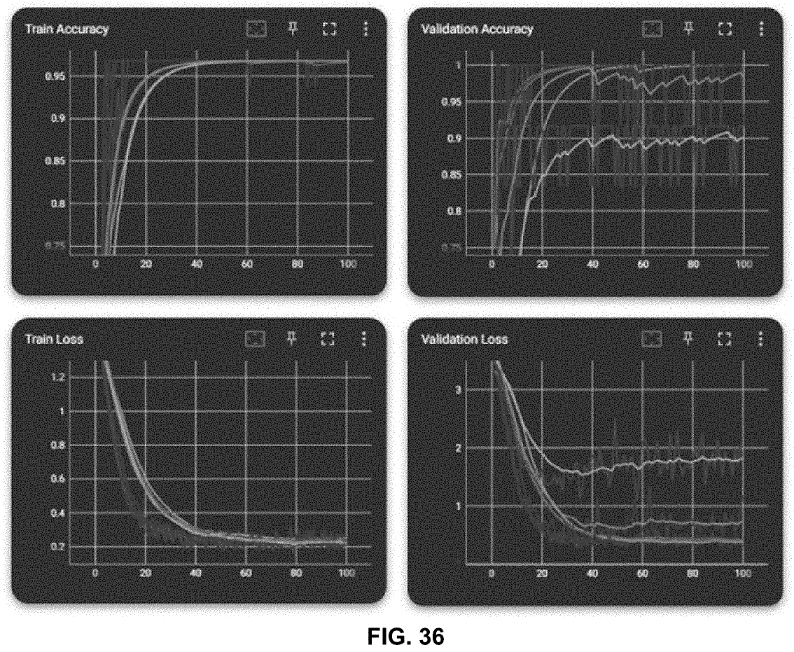

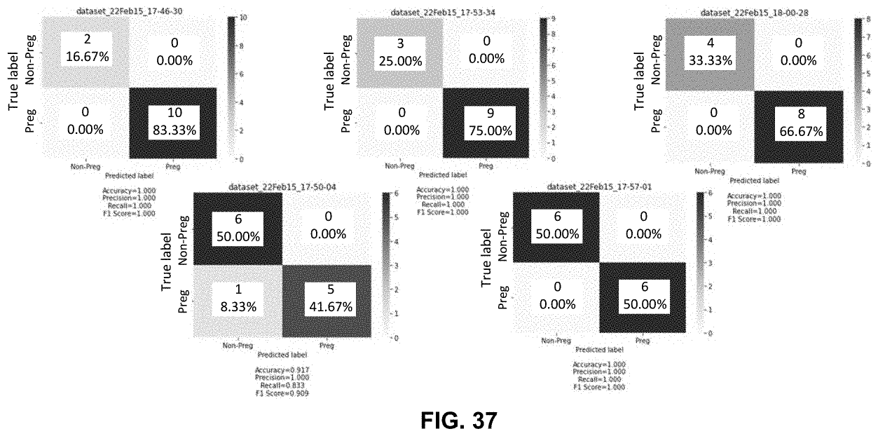

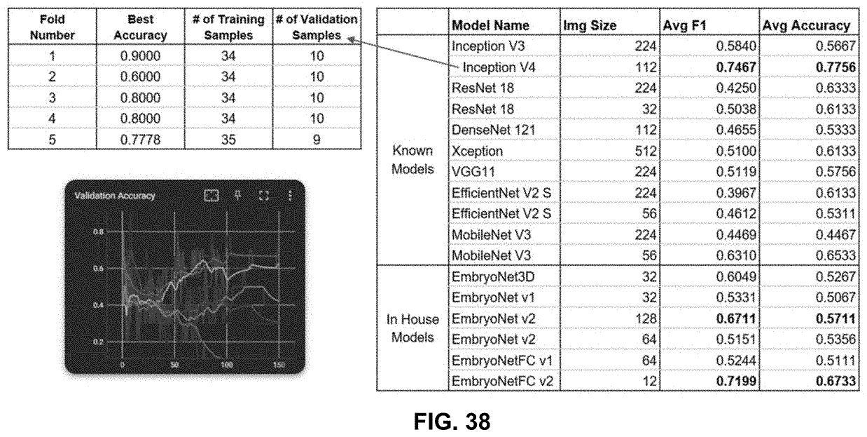

FIG. 36 shows training accuracy, validation accuracy, training loss, and validation loss plots for the EmbryoNet-FE model, in accordance with an embodiment. The first FE was trained for 1000 epochs (feature extraction). Subsequently, the head module (FC blocks) was replaced and the model was retrained with the FE weights frozen. Random augmentations were used during training, including flip, rotate, scale, and translation, as shown in FIG. 29. The dataset included 31 training data samples and 12 test data samples, including 25 pregnant cases and 18 non-pregnant cases. The average results were as follows: F1=98.18%, precision=100%, recall=96.67%, and accuracy=98.33%. FIG. 37 shows confusion matrices of results of the EmbryoNet-FE model on the pilot dataset, in accordance with an embodiment. FIG. 38 includes tabulated validation results on pilot data, comparing the various models and showing fold accuracies for the highest-performing models.

Embodiments described herein relate to use of microscopy in determining embryo viability. In some embodiments, a method can include accessing at a compute device, fluorescence lifetime imaging microscopy (FLIM) data set associated with a biological material. The biological material can include either an embryo or a gamete. The method further includes extracting a fluorescence photon arrival time from a subset of data from the FLIM data set. The method further includes estimating a likelihood that the biological material will produce a successful pregnancy and/or a live birth based on the fluorescence photon arrival time histogram and an estimation model that has been trained using artificial intelligence and labeled clinical training data. The method includes generating an output signal representing the estimated likelihood that the biological material will produce a successful pregnancy and/or a live birth.

In some embodiments, the gamete can include an oocyte.

In some embodiments, the gamete can include a sperm.

In some embodiments, the method can include training the estimation model using a plurality of fluorescence photon arrival time histograms of the FLIM data set. In some embodiments, the plurality of fluorescence photon arrival time histograms can include one of a raw intracellular FLIM histogram or a normalized intracellular FLIM histogram.

In some embodiments, the method can further include combining multiple fluorescence photon arrival time histograms from the plurality of fluorescence photon arrival time histograms of the FLIM data set.

In some embodiments, the fluorescence photon arrival time histogram can be a first fluorescence photon arrival time histogram. In some embodiments, estimating the likelihood that the biological material will produce a successful pregnancy and/or a live birth can be further based on a second fluorescence photon arrival time histogram.

In some embodiments, one of the first fluorescence photon arrival time histogram or the second fluorescence photon arrival time histogram can include one of a raw intracellular FLIM histogram or a normalized intracellular FLIM histogram.

In some embodiments, the method can further include parameterizing the fluorescence photon arrival time histogram using one of a decay model, phasor analysis, or principal component analysis, prior to estimating the likelihood that the biological material will produce a successful pregnancy and/or a live birth.

In some embodiments, the method can further include applying a physical model to data associated with the FLIM data set to generate an output, prior to estimating the likelihood that the biological material will produce a successful pregnancy and/or a live birth, wherein estimating the likelihood that the biological material will produce a successful pregnancy and/or a live birth is based on the output.

In some embodiments, the estimation model can include an artificial neural network.

In some embodiments, the method also includes performing a noise correction on the FLIM data set prior to estimating the likelihood that the biological material will produce a successful pregnancy and/or a live birth.

In some embodiments, the intracellular region can be spatially resolved such that different areas of the biological sample can be separately sampled.

In some embodiments, the method can further include partitioning the intracellular region to identify and sample one or more sub-cellular structures of the intracellular region.

In some embodiments, the FLIM data set can be generated by a system optimized to preferentially detect autofluorescence of nicotinamide adenine dinucleotide (NADH), using:

(i) one of a one-photon excitation wavelength between 305-385 nm or a two-photon excitation wavelength of between 720-760 nm; and

(ii) an emission bandpass filter having a lower cut-off between 400-450 nm and an upper cut-off between 450-485 nm.

In some embodiments, the FLIM data set can be generated by a system optimized to preferentially detect autofluorescence of flavin adenine dinucleotide (FAD), using:

(i) one of a one-photon excitation wavelength of between 380-500 nm or a two-photon excitation wavelength of between 800-950 nm; and

(ii) an emission bandpass filter having a lower cut-off of between 485-550 nm and an upper cut-off of about 550-650 nm.

In some embodiments, the FLIM data set can be generated by a system that does not use an emission bandpass filter.

In some embodiments, the FLIM data set can be generated by a system that uses multiple excitation wavelengths in succession, to obtain a hyperspectral representation of autofluorescence associated with the biological material.

In some embodiments, the FLIM data set can be generated by a system that uses a wavelength-splitting optic and a spectrographic detector to obtain a multispectral representation of the autofluorescence associated with the biological material.

In some embodiments, the method can further include updating the estimation model based on feedback generated during subsequent estimations.

In some embodiments, estimating the likelihood that the biological material will produce a successful pregnancy and/or a live birth is further based on spindle imaging. The spindle imaging can be performed via second harmonic imaging microscopy, e.g., generated with a non-linear pulsed laser.

In some embodiments, the estimation model has been trained using supervised artificial intelligence. In other embodiments, the estimation model has been trained using unsupervised artificial intelligence.

In some embodiments, the method also includes performing a signal filtering technique on the FLIM data set prior to estimating the likelihood that the biological material will produce a successful pregnancy and/or a live birth.

In some embodiments, the method can further include processing the FLIM data set to identify a subset of data from the FLIM data set that represents an intracellular region of the biological material.

In some embodiments, the method can further include using artificial intelligence to predict whether the embryo or the oocyte is aneuploid.

In some embodiments, the method can further include using artificial intelligence to predict whether the oocyte has matured.

In some embodiments, the method can further include validating the output signal. In some embodiments, the validating can be performed using a cross-validation method. In some embodiments, the cross-validation is a k-fold cross-validation.

In some embodiments, the biological material can include a plurality of sperm cells, and the method can further include comparing FLIM data of the sperm cells and selecting a viable sperm cell from the plurality of sperm cells.

In some embodiments, the biological material can include a plurality of sperm cells, and the method can further include analyzing the FLIM data of the plurality of sperm cells to determine a patient's overall sperm health.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisional s, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, the term “about” and “approximately” generally mean plus or minus 10% of the value stated, e.g., about 250 μm would include 225 μm to 275 μm, about 1,000 μm would include 900 μm to 1,100 μm.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. A method, comprising:
accessing, at a compute device, a fluorescence lifetime imaging microscopy (FLIM) data set associated with a biological material, the biological material including one of an embryo or a gamete;
extracting a fluorescence photon arrival time histogram from a subset of data from the FLIM data set;
estimating a likelihood that the biological material will produce a successful pregnancy and/or a live birth based on the fluorescence photon arrival time histogram and an estimation model that has been trained using artificial intelligence and labeled clinical training data; and
generating an output signal representing the estimated likelihood that the biological material will produce a one of a successful pregnancy or a live birth.

2. The method of claim 1, wherein the biological material includes the gamete, and the gamete includes an oocyte.

3. The method of claim 1, wherein the biological material includes the gamete, and the gamete includes a sperm.

4. The method of claim 1, further comprising:
training the estimation model using a plurality of fluorescence photon arrival time histograms of the FLIM data set.

5. The method of claim 4, further comprising using the trained model on non-training data to predict a patient's probability of producing a successful pregnancy and/or a live birth.

6. The method of claim 4, wherein the plurality of fluorescence photon arrival time histograms includes one of a raw intracellular FLIM histogram or a normalized intracellular FLIM histogram.

7. The method of claim 4, further comprising combining multiple fluorescence photon arrival time histograms from the plurality of fluorescence photon arrival time histograms of the FLIM data set.

8. The method of claim 1, wherein fluorescence photon arrival time histogram is a first fluorescence photon arrival time histogram, the estimating the likelihood that the biological material will produce a successful pregnancy and/or a live birth being further based on a second fluorescence photon arrival time histogram.

9. The method of claim 8, wherein one of the first fluorescence photon arrival time histogram or the second fluorescence photon arrival time histogram includes one of a raw intracellular FLIM histogram or a normalized intracellular FLIM histogram.

10. The method of claim 1, further comprising:
parameterizing the fluorescence photon arrival time histogram using one of a decay model, phasor analysis, or principal component analysis, prior to estimating the likelihood that the biological material will produce a successful pregnancy and/or a live birth.

11. The method of claim 1, further comprising applying a physical model to data associated with the FLIM data set to generate an output, prior to estimating the likelihood that the biological material will produce a successful pregnancy and/or a live birth, wherein the estimating the likelihood that the biological material will produce a successful pregnancy and/or a live birth is based on the output.

12. The method of claim 1, wherein the estimation model includes an artificial neural network.

13. The method of claim 1, further comprising performing a noise correction on the FLIM data set prior to estimating the likelihood that the biological material will produce a successful pregnancy and/or a live birth.

14. The method of claim 1, wherein the intracellular region is spatially resolved such that different areas of the biological sample can be separately sampled.

15. The method of claim 1, further comprising partitioning the intracellular region to identify and sample one or more sub-cellular structures of the intracellular region.

16. The method of claim 1, wherein the FLIM data set is generated by a system optimized to preferentially detect autofluorescence of nicotinamide adenine dinucleotide (NADH), using:
(i) one of a one-photon excitation wavelength between 305-385 nm or a two-photon excitation wavelength of between 720-760 nm; and
(ii) an emission bandpass filter having a lower cut-off between 400-450 nm and an upper cut-off between 450-485 nm.

17. The method of claim 1, wherein the FLIM data set is generated by a system optimized to preferentially detect autofluorescence of flavin adenine dinucleotide (FAD), using:
(i) one of a one-photon excitation wavelength of between 380-500 nm or a two-photon excitation wavelength of between 800-950 nm; and
(ii) an emission bandpass filter having a lower cut-off of between 485-550 nm and an upper cut-off of about 550-650 nm.

18. The method of claim 1, wherein the FLIM data set is generated by a system that does not use an emission bandpass filter.

19. The method of claim 1, wherein the FLIM data set is generated by a system that uses one of:
multiple excitation wavelengths in succession, to obtain a hyperspectral representation of autofluorescence associated with the biological material; or
a wavelength-splitting optic and a spectrographic detector to obtain a multispectral representation of the autofluorescence associated with the biological material.

20. The method of claim 1, wherein the estimating the likelihood that the biological material will produce a successful pregnancy and/or a live birth is further based on contextual data including one of patient-specific data, clinic-specific data, or a morphological image associated with the biological material.

21. The method of claim 1, further comprising updating the estimation model based on feedback generated during subsequent estimations.

22. The method of claim 1, wherein the estimating the likelihood that the biological material will produce a successful pregnancy and/or a live birth is further based on spindle imaging.

23. The method of claim 22, wherein the spindle imaging is via second harmonic imaging microscopy, generated with a non-linear pulsed laser.

24. The method of claim 1, wherein the estimation model has been trained using supervised artificial intelligence.

25. The method of claim 1, wherein the estimation model has been trained using unsupervised artificial intelligence.

26. The method of claim 1, further comprising performing a signal filtering technique on the FLIM data set prior to estimating the likelihood that the biological material will produce a successful pregnancy and/or a live birth.

27. The method of claim 1, further comprising:
processing the FLIM data set to identify a subset of data from the FLIM data set that represents an intracellular region of the biological material.

28. The method of claim 1, further comprising using artificial intelligence to predict whether the embryo or the gamete is aneuploid.

29. The method of claim 1, further comprising using artificial intelligence to predict whether the gamete has matured.

30. The method of claim 1, further comprising validating the output signal.

31. The method of claim 30, wherein the validating is performed using a cross-validation method.

32. The method of claim 31, wherein the cross-validation method includes k-fold cross-validation.

33. The method of claim 1, wherein the biological material includes a plurality of sperm cells, the method further comprising:
comparing FLIM data of the sperm cells; and
selecting a viable sperm cell from the plurality of sperm cells.

34. The method of claim 1, wherein the biological material includes a plurality of sperm cells, the method further comprising:
analyzing the FLIM data of the plurality of sperm cells to determine a patient's overall sperm health.

35. The method of claim 1, further comprising assessing an efficacy of a preparation medium based on at least one of the fluorescence photon arrival time histogram or the estimation model.
